(19) United States
(12) Patent Application Publication
(10) Pub. No.: US 2007/0082397 A1
(43) Pub. Date: Apr. 12, 2007

(54) Ex vivo progenitor and stem cell expansion for use in the treatment of disease of endodermally-derived organs

(76) Inventors: Arik Hasson, Kiryat-Ono (IL); Moshe Marikovsky, Mazkeret Batia (IL); Tony Peled, Mevasseret Zion (IL); Frida Grynspan, Mevasseret Zion (IL)

Correspondence Address: Martin D Moynihan;PRTSI Inc, P O Box 16446, Arlington, VA, 22215, US

(21) Appl. No.: 10/564,760

(22) PCT Filed: Jul. 15, 2004
(86) PCT No.: PCT/IL04/00644

§ 371 Date: Jul. 7, 2006

Related U.S. Application Data
(60) Provisional application No. 60/487,623, filed on Jul. 17, 2003.
(60) Provisional application No. 60/503,884, filed on Sep. 22, 2003.

(30) Foreign Application Priority Data
May 10, 2004 (IL) 161903

(51) Int. Cl. C12N 5/08

(52) U.S. Cl. 435/366; 435/370

(57) ABSTRACT

Methods of ex-vivo expansion of endodermally-derived and non-endodermally-derived progenitor and stem cells, expanded populations of renewable progenitor and stem cells and to their uses in therapeutic applications such as the production of endocrine hormones and the prevention and treatment of liver and pancreatic disease.

### FIELD AND BACKGROUND OF THE INVENTION

The present invention relates to methods of ex-vivo expansion of endodermally-derived and non-endodermally-derived progenitor and stem cells, to expanded populations of renewable progenitor and stem cells and to their uses. In particular, fetal and/or adult hepatic progenitor, and umbilical cord blood, bone marrow or peripheral blood derived stem cells expanded ex-vivo according to the methods of the present invention can be induced to express characteristics of endodermally-derived organs, such as liver and pancreas, and transplanted into appropriate solid organs for repopulation. The present invention further relates to therapeutic applications in which these methods and/or the expanded stern cells populations obtained thereby are utilized, such as the production of endocrine hormones and the prevention and treatment of liver and pancreatic disease.

Liver and Pancreatic Disease

Liver disease is a major concern of health care providers and policy makers worldwide, affecting millions of people each. Diseases of the liver are the third most common cause of death of Americans during their productive years. Three percent of the world's population, and approximately 5 million Americans, or greater than 2% of the US population, are currently infected with hepatitis C virus (HCV), making hepatitis C more prevalent than HIV. Between 8-10,000 Americans die each year due to HCV-related illness.

Liver disease includes primary liver disease such as primary biliary cirrhosis, hepatic cancer, primary sclerosing cholangitis, autoimmune chronic hepatitis, alcoholic liver disease (the most common cause of liver injury) and infectious disease such as hepatitis C, as well as secondary conditions such as the hepatic stage of parasitic infections (helminthes, etc), drug and chemical toxicity, many of which are life threatening. Furthermore, due to the liver's critical roles in metabolism and homeostasis, such as albumin synthesis, and unique vascularization, impaired liver function has serious consequences for the nervous, skeletal, digestive, endocrine and circulatory systems.

Liver failure can result from any type of liver disorder, including viral hepatitis, cirrhosis, and liver damage from alcohol or drugs such as acetaminophen. In acute liver failure, a person may go from being healthy to near death within a few days. In chronic liver failure, the deterioration in health may be very gradual until a dramatic event, such as bleeding varices (large, tortuous veins), occurs. Ultimately, liver failure is fatal if it is not treated or if the liver disease is progressive. Even after treatment, liver failure may be irreversible. In terminal cases, the person may die of kidney failure (hepatorenal syndrome), because liver failure can eventually lead to kidney failure. Liver transplantation, if performed soon enough, is a viable option (indeed, the only viable option in acute liver failure), but it is suitable for only a small number of people with liver failure. However, a liver transplant is not a treatment for certain diseases, such as some infections and types of cancer, because they likely will reoccur in the new organ. Furthermore, caring for the new liver is a lifelong commitment, requiring frequent blood tests and daily medications for the rest of the patient's life.

Currently there are over 14,000 people awaiting liver transplants in the United States, and many die while waiting for a compatible liver to become available. According to the UNOS Scientific Registry 1999 annual report, the median national waiting time in 1998 was 515 days, leading not only to increased mortality and morbidity, but also placing as extreme burden on the health care systems responsible for the support of transplant candidates in the interim. In addition, HCV (the virus that causes chronic liver disease), which affects a vast number of people, also results in cirrhosis and primary liver cancer (HCC) and many patients infected with HCV require liver transplantations.

Liver transplantation is severely hampered by the lack of available donors and therefore the search for new treatment modalities is of outmost importance. In addition to novel combinations of existing drugs (alpha interferon, ribavin, immune modulators and natural remedies), experimental therapies such as the bioartificial liver, gene therapy (for example, with Hepatocyte Growth Factor, HGF) and immunization are being explored. One of the strategies is to grow hepatocytes in culture to substitute organ transplantation. Hepatocytes have a remarkable capacity to proliferate in vivo but they grow poorly in vitro, making the preparation of significant numbers of hepatocytes suited for organ repopulation as yet unfeasible (Wick M, et al. ALTEX. 1997; 14(2): 51-56; Hino H, et al. Biochem Biophys Res Commun. Mar. 5, 1999;256(l): 184-91; and Tateno C, and Yoshizato K. Am J Pathol. 1996; 148(2): 383-92). Proliferation of primary rat hepatocytes in culture has been reported to be supported by the presence of bone marrow derived stromal cells (Mizuguchi et al, J Cell Phys 2001;1 89:106-119), by DMSO (Tang et al, Di Yi Jun Yi (Chinese) 2003, 23:106-19) and by CXC cytokines (U.S. patent application Ser. No. 6,719,969 to Hogaboam et al., which is incorporated by reference as if fully set forth herein), but systems of hepatocyte culture suitable for clinical applications are as yet unavailable.

Adult hepatic stem cells (oval cells) have also been considered for use in replacement therapy in acute liver failure. These oval cells express markers of immature liver cells, such as alpha-feto protein, and can differentiate into both hepatic epithelium and hepatocytes (Germain et al., 1988). Oval cells emerge during liver necrosis caused by chemical injury or when hepatocytes are treated with chemicals that block differentiation (Petersen, B. E., 2001); the origin of hepatic oval cells is unresolved (Wang et al, PNAS USA, 2003;100:11881-88). However, the numbers of oval cells naturally available from the adult liver is minimal, constituting a serious obstacle to the use of oval cells for transplantation. Thus, the use of oval cells as a viable therapeutic alternative to organ transplantation depends upon the development of novel and efficient techniques for ex-vivo oval cell expansion.

Pancreatic disease includes acute and chronic pancreatitis, hereditary pancreatitis, pancreatic cancer and diabetes. Of these, diabetes is the most significant and best studied.

Diabetes mellitus is the name for any condition characterized by chronic hyperglycemia and disturbances of carbohydrate, protein and fat metabolism. Diabetes results from a physiological malfunctioning of the pancreas, specifically, the secretory β cells of the islets of Langerhans. The increasing incidence of diabetes worldwide makes it a major public health concern. According to health authorities, more than 150 million people worldwide (approximately 8% of the population), and >18 million in the US, suffers from the disease. Worse, diabetes rates have been increasing in industrialized nations. In 1999, approximately 450,000 deaths occurred among people with diabetes aged 25 years and older. This figure represents about 19 percent of all deaths in the United States in people aged 25 years and older.

Complications of the chronic hyperglycemia of diabetes include endothelial damage, proliferative retinopathy, neuropathy, nephropathy, hypertension and ischemic heart disease. Diabetes is one of the leading causes of heart disease, stroke, kidney failure, blindness, and limb amputations and as such it is a drain on the economies of all industrial countries, accounting for more than $132 billion expenditure per year in the US alone. Current treatments cannot provide satisfactory solutions, so there is a need for new therapeutic approaches.

Type I juvenile) diabetes, which is responsible for 5-10% of the cases, is caused by autoimmune destruction of the pancreatic islet insulin-producing β cells. Type II diabetes, representing 90-95% of the cases, afflicts mostly people aged 45 and older and is thought to originate in an end-organ insensitivity to insulin, resulting in a decline in insulin synthesis and secretion by the pancreatic islet β cells. Type II diabetes is associated with obesity and a sedentary lifestyle, which are likely the causes of its increased incidence in modern society.

In its early form, type II diabetes may be managed with lifestyle changes in diet and exercise, and drugs that enhance insulin secretion. However, 40% of type II diabetic patients eventually require administration of large doses of insulin. Thus, the treatment of type I diabetes by insulin administration cannot avoid the long-term complications induced by daily cycles of hyper- and hypoglycemia, due to the difficulty of determining the exact insulin dosage required in changing physiological conditions. Although sophisticated technical solutions have often been proposed (indwelling insulin pumps, glucose-sensitive release mechanisms, etc) the ultimate clinical goal remains restoration of pancreatic function. Pancreas islet and organ transplantation have been attempted with inconsistent results. Attempts at pancreatic organ transplant have met with limited success (approximately 50% survival at 15 years), and even less for islet cell transplants, which have been largely unsuccessful due to the destruction of transplanted β cells in recurring episodes of autoimmune inflammation. Furthermore, pancreas and islet transplant suffer from the general low availability of suitable matched donated organs, making waiting lists very long. Thus, treatment for type I diabetes is likely to be the replacement of diseased β cells with intact β cells through transplantation. Such therapy can also be applicable to type II diabetes which in its early stages is presently managed through a combination of drugs that increase insulin secretion, and lifestyle changes in diet and exercise.

Thus, there is clearly a need for an abundant source of liver and pancreas cells, or liver and pancreas cell progenitors, suitable for implantation and repopulation of these endodermally-derived organs, for treatment or prevention of liver and pancreatic disease. Once an abundant source of cells is developed, methods will be developed to transplant them in a way that will avoid their destruction by recurring autoimmunity.

Expansion of Stem and Progenitor Cell Populations:

While many methods for stimulating proliferation of stem and progenitor cell populations have been disclosed [see, for example, Czyz et al, Biol Chem 2003; 384:1391-409; Kraus et al., (U.S. Pat. No. 6,338,942, issued Jan. 15, 2002); Rodgers et al. (U.S. Pat. No. 6,335,195 issued Jan. 1, 2002); Emerson et al. (Emerson et al., U.S. Pat. No. 6,326,198, issued Dec. 4, 2001) and Hu et al. (WO 00/73421 published Dec. 7, 2000) and Hariri et al (US Patent Application No. 20030235909)] few provide for reliable, long-term expansion, without the accompanying differentiation that naturally occurs with growth of stem or progenitor cells in culture.

Hematopoietic Cellular Differentiation

Much of the knowledge regarding the pathways and mechanisms underlying cellular differentiation has been extracted by careful studies on the hematopoietic system. Hematopoietic stem cells (HSCs) are responsible for maintaining normal production of blood cells (hematopoiesis), in the face of continuous cell loss to programmed cell death (apoptosis) and removal of aging cells by the reticulo-endothelial system. In the event of stress such as trauma, proper hematopoietic functioning allows release of cellular reservoirs from the marrow, downregulation of apoptosis and loss of mature cells, and enhanced proliferation of HSCs and progenitors. Such modulation of the hematopoietic system is achieved through the concerted actions of cytokines (which facilitate cell-cell and cell-matrix interactions), chemokines, and extracellular matrix (ECM) components. A single HSC can give rise to all types of hematopoietic cells, and is found in very low numbers predominantly in the bone marrow (although HSCs are also found in umbilical cord blood (UBC) and other tissues). Studies characterize human HSCs as small quiescent cells that express high levels of the surface glycoprotein CD34 (CD34+), and low or undetected levels of markers such as CD33, CD38, thy-1, and CD71, which designate a more mature progenitor population. CD34+CD38− cells (which represent <10% of the limited CD34+ cell population) can give rise to both lymphoid and myeloid cells in vitro, repopulate immune-compromised mice to high degrees, and appear critical to hematopoietic recovery of patients receiving autologous blood cell transplantation. In line with their ascribed role, noticeable levels of telomerase, an enzyme essential for genomic integrity and cellular proliferation, can be found in CD34+CD38− cells. Despite heightened interest in the use of these cells as therapeutic agents, population scarcity as well as poor ex vivo expansion abilities hindered their use in a clinical setting. Currently used methods of ex vivo expansion are growth of mononuclear cells, with or without prior selection for CD34 expression, with a combination of early and late growth factors; with or without serum; with or without a stromal cell layer; in stationary or rapid medium exchanged cultures; or utilizing bioreactors. In all the abovementioned systems, significant accumulation of intermediate and late progenitors is achieved, with little if any expansion of the CD34+CD38− subpopulation. Such failure in expansion of the early hematopoietic fraction is detrimental for any prospect of utilizing these expanded cultures in transplantation experiments. Current efforts are targeted at developing expansion techniques that un-couple proliferation from differentiation; such techniques may support the expansion of CD34+CD38− cells for a prolonged period without the concomitant progression of the differentiation program.

Up until recently, expansion of renewable stem cells has been achieved either by growing the stem cells over a feeder layer of fibroblast cells, or by growing the cells in the presence of the early acting cytokines thrombopoietin (TPO), interleukin-6 (IL-6), an FLT-3 ligand and stem cell factor (SCF) (Madlambayan G J et al. (2001) J Hematother Stem Cell Res 10: 481, Punzel M et al. (1999) Leukemia 13: 92, and Lange W et al. (1996) Leukemia 10: 943). While expanding stem cells over a feeder layer results in vast, substantially endless cell expansion, expanding stem cells without a feeder layer, in the presence of the early acting cytokines listed above, results in an elevated degree of differentiation (see Leslie N R et al. (Blood (1998) 92: 4798), Petzer A L et al. (1996) J Exp Med Jun 183: 2551, Kawa Y et al. (2000) Pigment Cell Res 8: 73).

Recently, however, methods for feeder-layer free expansion of stem cells ex-vivo have been disclosed. PCT IL99/00444 to Peled et al., filed Aug. 17, 1999, which is incorporated by reference as if fully set forth by reference herein, and from which the present invention derives priority, disclosed methods of imposing proliferation yet restricting differentiation of stem and progenitor cells by treating the cells with chelators of transitional metals. While reducing the invention to practice, they uncovered that heavy metal chelators having a high affinity for copper, such as tetraethylpentamine (TEPA), greatly enhanced the fraction of CD34⁺ cell and their long-term clonability in cord-blood-derived, bone marrow-derived, and peripheral blood derived stem and progenitor cells, grown without a feeder layer. Facilitation of proliferation while inhibiting differentiation was also observed in erythroid progenitor cells, cultured mouse erythroleukemia cells, embryonal stem cells, and hepatocytes in primary hepatocyte culture treated with TEPA.

PCT IL03/00062, also to Peled et al., filed Jan. 23, 2003, which is incorporated by reference as if fully set forth herein, and from which the present invention derives priority, discloses a similar effective promotion of long term ex vivo stem cell proliferation, while inhibiting differentiation, using TEPA-Cu chelates as well as the chelator TEPA. Surprisingly, this effect of TEPA and TEPA-chelates was also demonstrated using as a starting population an un-selected peripheral mononuclear fraction. The results described there-in clearly show that stem and progenitor hematopoietic cells may be substantially expanded ex vivo, continuously over at least 12 weeks period, in a culture of mixed (mononuclear fraction) blood cells, with no prior purification of CD₃₄⁺ cells.

PCT IL 03/00064, also to Peled et al., filed Jan. 26, 2003, which is incorporated by reference as if fully set forth herein, and from which the present invention derives priority, teaches the ex-vivo expansion and inhibition of hematopoietic stem and progenitor cells using conditions and various molecules that interfere with CD38 expression and/or activity and/or with intracellular copper content, for inducing the ex-vivo expansion of hematopoietic stem cell populations. The small molecules and methods include linear polyamine chelators and their chelates, nicotinamide, a nicotinamide analog, a nicotinamide or a nicotinamide analog derivative or a nicotinamide or a nicotinamide analog metabolite, a PI 3-kinase inhibitor, conditions for reducing a capacity of the hematopoietic mononuclear cells in responding to retinoic acid, retinoids and/or Vitamin D and reducing the capacity of the cell in responding to signaling pathways involving PI 3-kinase.

Surprisingly, the inventors also showed that exposure of hepatocytes in primary culture to the small molecules, and conditions described hereinabove stimulated hepatocyte proliferation, greatly expanding the fraction of undifferentiated and immature hepatocytes (as determined by a-feto-protein expression, OC3 marker expression and oval cell morphology). Thus, using the methods described, adult stem and progenitor cells of hematopoietic and non-hematopoietic origin can provide expanded populations of cells for transplantation into endodermally derived organs.

PCT IL 03/00681, also to Peled et al, filed Aug. 17, 2003, which is incorporated by reference as if fully set forth herein, and from which the present invention derives priority, discloses methods of ex-vivo expanding a population of hematopoietic stem cells present, even as a minor fraction, in hematopoietic mononuclear cells, without first enriching the stem cells, while at the same time, substantially inhibiting differentiation of the hematopoietic stem cells. Cells thus expanded can be used to efficiently provide ex-vivo expanded populations of hematopoietic stem cells without prior enrichment of the hematopoietic mononuclear cells for stem cells suitable for hematopoietic cell transplantation, for genetic manipulations for cellular gene therapy, as well as in additional application such as, but not limited to, adoptive inmmunotherapy, implantation of stem cells in an in vivo cis-differentiation and trans-differentiation settings, as well as, ex-vivo tissue engineering in cis-differentiation and trans-differentiation settings.

PCT IL 2004/000215, also to Peled et al., filed Mar. 4, 2004, which is incorporated by reference as if fully set forth herein, and from which the present invention derives priority, furter demonstrated the self-renewal of sten/early progenitor cells, resulting in expansion and inhibition of differentiation in stem cells of hematopoietic origin and non-hematopoietic origin by exposure to low molecular weight inhibitors of PI 3-kinase, disruption of the cells' PI 3-K signaling pathways.

Organ Repopulation by Stem and Progenitor Cells

Trans-differentiation of cells, to novel cell types, is described in the literature. For example, Levesque et al (U.S. Pat. No. 6,087,168, incorporated herein by reference) disclose methods for production of neuronal cells from non-neuronal epidermal basal cells by transgenic expression of a neurogenic transcription factor, anti-sense suppression of negative regulators of neuronal differentiation, and addition of retinoids and neurotrophins to the medium. Non-endodermally-derived cells, such as hematopoietic progenitor cells derived from cord blood (UCB), bone marrow or peripheral blood can also be converted into sources of liver or pancreatic cells. Mononuclear UCB cells produce albumin upon cultivation in the presence of hepatic growth factors such as FGF-1, FGF-2, LIF and OSM and have the capacity to home to the liver and develop into functional hepatocytes when transplanted into liver-injured severe combined immuno-deficient (SCID) mice (Kakinuma et al., 2003). However, no expansion of the cultured cells was reported. Bone marrow progenitor cells can also develop into active hepatocytes in-vivo under certain circumstances (Petersen et al, 1999; Theise et al., 2000). Cultured BM mononuclear cells in the presence of HGM, Fetal Bovine Serum (FBS), Human Growth Factor (HGF) and Epidermal Growth Factor (EGF) produce hepatocytes-like colonies that are also albumin positive. A subset of the adult rat bone marrow population has been shown to be positive for hepatocyte growth factor (HGF), receptor c-Met, and α-fetoprotein alongside with hematopoietic stem cell markers such as CD34, Thy-1 and c-kit (Miyazaki et al, 2002). Furthermore, cells expressing albumin, CK18 and HNF (previously referred to as HNF-1, LPB1, and APF) can be generated from primitive, multipotent adult bone marrow-derived progenitor cells. These cells can acquire a hepatocyte phenotype and functional characteristics of hepatocytes (Schwartz et al., 2002).

Embryonic cells can be an alternative source of hepatic cells. Mouse embryonic cells have the potential to differentiate into hepatocytes in vitro in the presence of growth factors for hepatic maturation (Hamazaki et al., 2001). Recently, human Embryonic Stem Cells (hESC) have been shown to differentiate to hepatic cells by the addition of sodium butyrate (Rambhatla et al., 2003), suggesting that cultivation of hESC or other pluripotent stem cells in large quantities may provide an alternative source for hepatic tissue engraftment. However, the cultivation of such stem or progenitor cells in significant quantities, while inhibiting spontaneous differentiation remains a formidable task.

Since mature β cells do not divide much in vitro, oncogene expression has been used to force their replication. Studies involving expression of oncogenes in β cells of transgenic mice resulted in the development of β-cell lines, which proved valuable for investigating β-cell function and gene expression (1). However, induction of forced replication of post-mitotic β cells ultimately leads to impaired insulin production and impaired secretion in these cell lines. The phenotypic instability, as well as the uncontrolled growth, has made this approach incompatible with a therapeutic application and demands of regulatory authorities.

The alternative to expansion of mature β cells is differentiation of stem or progenitor cells into surrogate β cells. Stem cells possess a natural replication capacity in tissue culture. Ianus et al. (J Clin. Invest. 2003; 11 1:843), using bone marrow cells from transgenic GFP mice, demonstrated that BM cells injected into irradiated host animals can repopulate the pancreatic islets with GFP-expressing cells. When allowed to spontaneously differentiate, both murine and human embryonic stem (ES) cells give rise to a small percentage of insulin-producing cells (6,7). Initial efforts were directed towards isolation of these relatively rare cells using selection procedures. Subsequently, protocols were developed to increase the fraction of ES cells which develop into insulin-producing cells. McKay and co-workers selected nestin-positive neuroendocrine precursor cells, which developed from mouse ES cells, and utilized combinations of soluble factors to promote their differentiation in tissue culture into islet cell types (8). Hori et al. treated mouse ES cells with inhibitors of phosphoinositide 3-kinase, thereby generating cells that produced significant insulin levels and released it in response to glucose (9). While insulin-producing cells developed from ES cells were able to normalize glycemia in mice, the ability of such cells derived from human ES cells to replace the function of differentiated β cells in humans remains unknown.

In addition to stem cells derived from early-age embryos, evidence suggests that many fetal and adult tissues contain immature cells, which are responsible for tissue renewal. Such cells maintain a replicative capacity and an ability to differentiate into a number of cells types. The most obvious place to look for cells that can potentially differentiate into insulin-producing cells is the pancreas. Duct cells can form islet-like structures in culture (10,11), however they are difficult to expand. The isolation and characterization of the pancreatic islet stem cells remains a goal for future efforts.

The successful replacement of beta cells depends on the availability of human organs supply and the protection of the transplanted cells from immune destruction. Even if immunosuppressive therapy can minimize immune rejection, the shortage of donors is such that it will not be possible to meet the expected demand (Soria et al., 2001). The limited supply of human pancreatic glands makes obvious the need to develop alternative approaches to overcome the shortage of cells by searching for new cell sources and expansion technologies.

Human embryonic stem cells (hES-H9) have been induced to differentiate into insulin producing cells (Assady et al., 2001), but this was restricted to a small subset of the cells. Genetic manipulation of embryonic stem cells has been performed in mouse embryonic lines that after clonal selection were shown to normalize glycemia in streptozotocin-induced diabetic mice (Soria et al., 2000).

The use of human embryonic stem cells, although promising, presents numerous practical problems, such as the obligatory feeder cell layer on which the available embryonic stem cell lines grow, immunogenic and tumorogenic issues, and ethical considerations.

Hepatic cells from fetal origin or adult stem hepatic cells (oval cells) are an alternative source for pancreatic-insulin producing cells. Highly purified adult rat hepatic oval cells can trans-differentiate into pancreatic endocrine hormone-producing cells when cultured in a high-glucose environment (Yang et al., 2002). Fetal human progenitor liver cells were induced into insulin-producing cells after expression of the pancreatic duodenal homeobox (PDX1) gene, and their replication capacity enhanced by the additional introduction of the gene of the catalytic subunit of human telomerase (Zalzman et al., 2003). US Patent Application No. 20030219894 to Seino et al., incorporated herein by reference, discloses methods of transdifferentiation of mammalian fetal hepatocytes by expression of PDX-1 and/or NeuroD, a transcription factor important for pancreatic development. US Patent Application Nos. 20020164308 and 20020068045 to Reubinoff et al., incorporated herein by reference, disclose the directed differentiation of human embryonic stem cells, grown on fibroblast feeder layers, into neurogenic progenitors, and their transdifferentiation, with non-neural growth and differentiation factors.

US Patent Application No. 20030185805 to Dai et al., incorporated herein by reference, discloses the use of human dermal skin CD34⁺ cells for the production of tissues and organs for repopulation and implantation, including endodermally derived organs such as liver and pancreas. Another US Patent Application by the same inventors, No. 20020068051 to Dai et al., incorporated herein by reference, discloses the transdifferentiation of human skin fibroblasts to liver cells using liver extracts containing “Signalplexes” responsible for directing differentiation of pluripotent progenitor cells. US Patent Application No. 20020068046, also to Dai et al., further discloses the transdifferentiation of pancreatic fibroblasts by “Signalplexes”. However, no “Signalplexes” are isolated or identified, and only hypothetical experiments, lacking results, are presented.

Israeli Patent Application No. 161903, filed May 10, 2004, to Peled et al., which is incorporated by reference as if fully set forth herein, and from which the present invention derives priority, discloses the expansion of endodermal- and non-endodermally derived progenitor and stem cells for transplantation and the repopulation of endodermal organs.

The successful development of a cell therapy for the treatment of liver and pancreatic diseases based on embryonic, fetal, adult stem cells and/or umbilical cord blood, bone marrow or peripheral blood stem cells depends on the availability of sufficient cell number for treatment. Based on the above descriptions, it is clear that there is thus a widely recognized need for, and it would be highly advantageous to have, methods enabling ex-vivo expansion of endodermally-derived and non-endodermally-derived progenitor and stem cells yielding large numbers of these cell populations for transplantation into appropriate solid organs for repopulation.

### SUMMARY OF THE INVENTION

The present invention discloses methods of ex-vivo expansion of adult and/or fetal, endodermally-derived and non-endodermally derived stem and/or progenitor cells origins, to expanded populations of renewable progenitor and stem cells, and methods of the use thereof for transplantation and repopulation of endodermally-derived organs.

The novel methods disclosed herein may be used for ex-vivo expansion of endodermally-derived and non-endodermally derived stem cells, and for transdifferentiation of non-endodermally derived stem cells, resulting in renewable populations of stem cells having endodermal cell character, for transplantation and repopulation of solid, endodermally derived organs such as liver and pancreas.

While reducing the present invention to practice, it was unexpectedly found that ex-vivo expansion of stem and progenitor cells using a unique culturing system, and the addition of hepatic or pancreatic factors, resulted in large populations of implantable stem and progenitor cells, having pancreatic or hepatic character. Surprisingly, implantation of these cells into the pancreas of streptozotocin-diabetic SCID mice resulted in repopulation of the diseased islets, and reversal of the diabetic symptoms. Thus, it is expected that this novel method of ex-vivo expansion of renewable endodermal stem and progenitor cells can be used for therapeutic and clinical applications as is further detailed hereinunder.

According to one aspect of the present invention there is provided a method of enhancing function of an endodermally derived organ in a subject in need thereof, the method effected by: (a) obtaining a population of cells comprising stem and/or progenitor cells; (b) culturing the stem and/or progenitor cells ex-vivo under conditions allowing for cell proliferation and, at the same time, culturing said cells under conditions selected from the group consisting of: (i) conditions reducing expression and/or activity of CD38 in the cells; (ii) conditions reducing capacity of the cells in responding to signaling pathways involving CD38 in the cells; (iii) conditions reducing capacity of the cells in responding to retinoic acid, retinoids and/or Vitamin D in the cells; (iv) conditions reducing capacity of the cells in responding to signaling pathways involving the retinoic acid receptor, the retinoid X receptor and/or the Vitamin D receptor in the cells; (v) conditions reducing capacity of the cells in responding to signaling pathways involving PI 3-kinase; (vi) conditions wherein the cells are cultured in the presence of nicotinamide, a nicotinamide analog, a nicotinamide or a nicotinamide analog derivative or a nicotinamide or a nicotinamide analog metabolite; (vii) conditions wherein the cells are cultured in the presence of a copper chelator; (viii) conditions wherein the cells are cultured in the presence of a copper chelate; (ix) conditions wherein the cells are cultured in the presence of a PI 3-kinase inhibitor; thereby expanding the stem and/or progenitor cells while at the same time, substantially inhibiting differentiation of the stem and/or progenitor cells ex-vivo; and (c) implanting the cells in an endodermally-derived organ of the subject.

According to further features in preferred embodiments of the invention described below the method ftrter comprising monitoring function of said endodermally-derived organ in said subject.

According to another aspect of the present invention there is provided a method of expanding and transdifferentiating a population of non-endodermally derived stem cells into stem cells having an endodermal phenotype, the method comprising: (a) obtaining a population of cells comprising stem and/or progenitor cells; (b) culturing the stem and/or progenitor cells ex-vivo under conditions allowing for cell proliferation and, at the same time, culturing said cells under conditions selected from the group consisting of: (i) conditions reducing expression and/or activity of CD38 in the cells; (ii) conditions reducing capacity of the cells in responding to signaling pathways involving CD38 in the cells; (iii) conditions reducing capacity of the cells in responding to retinoic acid, retinoids and/or Vitamin D in the cells; (iv) conditions reducing capacity of the cells in responding to signaling pathways involving the retinoic acid receptor, the retinoid X receptor and/or the Vitamin D receptor in the cells; (v) conditions reducing capacity of the cells in responding to signaling pathways involving PI 3-kinase; (vi) conditions wherein the cells are cultured in the presence of nicotinamide, a nicotinamide analog, a nicotinamide or a nicotinamide analog derivative or a nicotinamide or a nicotinamide analog metabolite; (vii) conditions wherein the cells are cultured in the presence of a copper chelator; (viii) conditions wherein the cells are cultured in the presence of a copper chelate; (ix) conditions wherein the cells are cultured in the presence of a PI 3-kinase inhibitor; thereby expanding the stern and/or progenitor cells while at the same time, substantially inhibiting differentiation of the stem and/or progenitor cells ex-vivo; and (c) inducing enrichment of said stem/progenitor cells for stem cells expressing endodermal cell markers, thereby expanding and transdifferentiating a population of non-endodermal stem cells into endodermal stem cells.

According to frrther features in preferred embodiments of the invention described below the stem and/or progenitor cells are derived from a source selected from the group consisting of hematopoietic cells, umbilical cord blood cells, G-CSF mobilized peripheral blood cells, bone marrow cells, hepatic cells, pancreatic cells, neural cells, oligodendrocyte cells, skin cells, gut cells, embryonal stem cells, muscle cells, bone cells, mesenchymal cells, chondrocytes and stroma cells.

According to yet further features in preferred embodiments of the invention described below step (b) is followed by a step comprising inducing ex-vivo enrichment of the stem/progenitor cells for stem cells having an endodermal cell phenotype.

According to still furher features in preferred embodiments of the invention described below the inducing is effected by providing at least one hepatic growth factor and/or sodium butyrate.

According to ftrer features in preferred embodiments of the invention described below the hepatic growth factor is selected from the group consisiting of FGF-1, FGF-2, LIF, OSM, HGM, FBS, HGF, EGF, and SCF.

According to yet further features in preferred embodiments of the invention described below the method further comprising the step of selecting a population of stem cells enriched for hematopoietic stem cells.

According to still further features in preferred embodiments of the invention described below the selection is affected via CD34.

According to further features in preferred embodiments of the invention described below the method further comprising the step of selecting a population of stem cells enriched for early hematopoietic sten/progenitor cells.

According to yet further features in preferred embodiments of the invention described below the selection is affected via CD133.

According to still further features in preferred embodiments of the invention described below step (b) is followed by a step comprising selection of stem and/or progenitor cells.

According to yet further features in preferred embodiments of the invention described below the selection is affected via CD 133 or CD 34.

According to further features in preferred embodiments of the invention described below the endodermally-derived organ is a liver, an intestine or a pancreas.

According to yet further features in preferred embodiments of the invention described below the providing the conditions for cell proliferation is effected by providing the cells with nutrients and cytokines.

According to still further features in preferred embodiments of the invention described below the cytokines are selected from the group consisting of early acting cytokines and late acting cytokines.

According to further features in preferred embodiments of the invention described below the early acting cytokines are selected from the group consisting of stem cell factor, FLT3 ligand, interleukin-6, thrombopoietin and interleukin-3.

According to still further features in preferred embodiments of the invention described below the late acting cytokines are selected from the group consisting of granulocyte colony stimulating factor, granulocyte/macrophage colony stimulating factor and erythropoietin.

According to further features in preferred embodiments of the invention described below the late acting cytokine is granulocyte colony stimulating factor.

According to yet further features in preferred embodiments of the invention described below the subject is a human.

According to still further features in preferred embodiments of the invention described below the stem and/or progenitor cells are genetically modified cells.

According to further features in preferred embodiments of the invention described below the stem and/or progenitor cells are derived from the subject.

According to yet further features in preferred embodiments of the invention described below the inhibitors of PI 3-kinase are wortnannin and/or LY294002.

According to further features in preferred embodiments of the invention described below the endodermal cell markers are selected from the group consisting of insulin, glucagon, somatostatin, pancreatic polypeptide, Pdx-1, pancreatic enzymes, C-peptide, albumin, CK18, CK 19, HNF, THY-1 receptor, c-Met receptor and c-kit.

According to still further features in preferred embodiments of the invention described below, the culturing of the stem and/or progenitor cells further comprises co-culturing said stem and/or progenitor cells with endodermally-derived organ tissue.

According to yet another aspect of the present invention there is provided a therapeutic ex vivo cultured stem cell population comprising non-endodermally-derived cells expanded and transdifferentiated according to the abovementioned methods.

According to fuer features in preferred embodiments of the invention described below the cell population is provided in a culture medium comprising at least one hepatic growth factor and/or sodium butyrate.

According to yet further features in preferred embodiments of the invention described below the cell population is isolated from the medium.

According to another aspect of the present invention there is provided a pharmaceutical composition comprising the cell population of and a pharmaceutically acceptable carrier.

According to further features in preferred embodiments of the invention described below the pharmaceutical composition comprising the cell population and a pharmaceutically acceptable carrier.

According to yet another aspect of the present invention there is provided a method of producing an endocrine hormone, the method comprising the abovementioned method and further comprising the step of continuing to culture the transdifferentiated cells in said medium, whereby an endocrine hormone may be produced.

According to further features in preferred embodiments of the invention described below the endocrine hormone is selected from the group consisting of insulin, glucagon and somatostatin.

According to another aspect of the present invention there is provided endocrine hormones produced by the abovementioned method.

According to another aspect of the present invention there is provided the abovementioned method used for treating or preventing a liver or pancreatic disease.

According to further features in preferred embodiments of the invention described below the liver disease is selected from the group consisting of primary biliary cirrhosis, hepatic cancer, primary sclerosing cholangitis, autoimmune chronic hepatitis, alcoholic liver disease, infectious hepatitis, parasitic hepatic disease, steatohepatitis and hepatic toxicity.

According to further features in preferred embodiments of the invention described below the pancreatic disease is selected from the group consisting of acute pancreatitis, chronic pancreatitis, hereditary pancreatitis, pancreatic cancer, and diabetes.

The present invention successfully addresses the shortcomings of the presently known configurations by providing a method of propagating cells, yet delaying their differentiation by interference with CD38 or PI 3-kinase expression, activity, and/or PI 3-kinase signaling.

The present invention further successfully addresses the shortcomings of the presently known configurations by enabling expansion of endodermally-derived and non-endodermally-derived progenitor and stem cells yielding large numbers of these cell populations for transplantation into appropriate solid organs for repopulation.

Additional features and advantages of the methods of cell preparations and methods of treatment according to the present invention will become apparent to the skilled artisan by reading the following descriptions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1C are photomicrographs depicting the effect of TEPA on expansion of hepatocyte progenitor cells (oval cells) derived from adult rat liver. Hepatocytes harvested from female VLVC mice, as described herein, by grinding and sieving, were plated at a density of 4-×10⁴/ml in F12 containing 15 mM Hepes, 0.1% glucose, 10 mM sodium bicarbonate, 100 units/ml penicillin-streptomycin, glutamine, 0.5-units/ml insulin, 7.5 mcg/ml hydrocortisone, and 10% fetal bovine serum, EGF (10 ng/ml), and HOF (20 ng/mi). FIG. 1A—No additions (control); FIGS. 1B—medium supplemented with 10 μM AGN 194310, 1C—medium supplemented with TEPA 10 μM. Note the abundance of oval cells in the TEPA- and AGN 194310-treated cultures.

FIG. 2 is a graphic representation of the restoration of pancreatic islet function by transplanted expanded Cord Blood cells in STZ-diabetic SCID mice. Seven SCID/SCID/bg/bg, STZ-diabetic mice received ex-vivo expanded stem cells in intra-pancreatic injection, two with re-selected CD133⁺ cells (squares, 5108P and 5104P), four with the fraction of total nucleated cells (TNCs)(circles, 5105, 5111, 5115 and 5116). One mouse received only PBS vehicle (control) (triangle, 5117C). Blood glucose was determined at different times post-implantation. Note the complete restoration of euglycemia with CD133⁺ cells (5108P), and the significant reduction of hypoglycemia with TNCs (5115 and 5116).

FIG. 3 is a graphic representation of the restoration of pancreatic function and response to a glucose load in STZ diabetic mice with transplanted expanded Cord Blood cells. Mice transplanted with expanded Cord Blood cells as in FIG. 2, 49 days after transplantation, fasted for 6 hrs, received lmg per gram glucose I.P., and blood glucose levels were monitored in tail vein blood at the indicated intervals. Note the total restoration of normal pancreatic function by transplantation of CD133⁺ cells (5108P, squares), and partial restoration by transplantation of TNCs (5115 and 5116, circles).

FIG. 4 is a graphic representation of restoration of pancreatic islet function by transplanted, expanded co-cultured hEnd stem cells in STZ-diabetic SCID mice. Two SCID/SCID/bg/bg, STZ-diabetic mice received CD133+, ex-vivo expanded stem cells that were cultured in hEndSC conditions, co-cultured with injured murine liver tissue, returned to hEndSC conditions, and transplanted in intra-pancreatic injection (circles, 554, 552). Two mice received only PBS vehicle (control) (square, 555, 552). Blood glucose was determined at different times post-implantation. Note the complete and rapid restoration of euglycemia with the co-cultured hEndSC (554 and 522).

### DESCRIPTION OF THE PREFERRED EMBODIMENTS

The present invention is of methods of ex-vivo expansion of endodermally-derived and non-endodermally-derived progenitor and stem cells, to expanded populations of renewable progenitor and stem cells and to their use for transplantation into appropriate solid organs for repopulation. In one embodiment, the invention facilitates the efficient establishment of ex-vivo expanded populations of stem and/or progenitor cells derived from cord blood, bone marrow, peripheral blood or endodermal organ cells suitable for transplantation into endodermally-derived organs. Specifically, the ex-vivo expanded cells can be used to treat diseases of, and restore function in endodermally derived organs such as liver and pancreas. The methods of the invention can also be used for applications in cellular gene therapy of transplanted, repopulated organs. Additional applications may include, but are not limited to, treatments for multiple diseases, such as, for example, acute or chronic liver failure and type I or type II diabetes, ex-vivo trans-differentiation and implantation of stem and/or progenitor cells, ex vivo tissue engineering and ex-vivo production of pancreatic hormones.

While reducing the present invention to practice, it was unexpectedly found that non-endodermally derived stem cells, such as the total nucleated cell fraction of cord blood cells, when induced to proliferate and expand ex-vivo without differentiation, can repopulate and restore function to injured and diseased endodermal organs, by direct implantation into the organs. In similar experiments it was found that ex-vivo expanded, cord blood CD133⁺ cells can also repopulate and restore function to the injured organ; hence, the repopulation is not dependent on differentiation during expansion of cells to endodermal progenitor cell types. As is described in the Background section, presently there is no disclosed technology by which to expand non-endodermally-derived stem cells for implantation to endodermal organs.

Furthermore, primary hepatocyte cultures incubated with retinoic acid receptor antagonists, or linear polyarines such as TEPA, revealed an increase in the proportion of oval cells, hence inducing the proliferation of early hepatocyte populations. TEPA or RAR antagonist-treated hepatocyte cultures persisted for at least 3 weeks in culture, a finding in stark contrast to previous data indicating a near impossibility in growing primary hepatocytes for extended periods of time in culture (Wick M, et al. ALTEX. 1997; 14(2): 51-56; Hino H, et al. Biochem Biophys Res Commun. Mar. 5, 1999;256(1): 184-91; and Tateno C, and Yoshizato K. Am J Pathol. 1996; 148(2): 383-92). Supplementation with growth factors alone was insufficient to stimulate hepatocyte proliferation, only RAR antagonist or TEPA treatment of hepatocyte cultures resulted in the proliferation of early hepatocyte populations and in their persistence in culture, evident even following first and second passages.

Thus, adult or fetal endodermally-derived cells can also be ex-vivo expanded in the context of the present invention, providing an abundant source of cell populations for treatment of conditions and diseases of endodermal organs by transplantation and repopulation.

The principles and operation of the present invention may be better understood with reference to the drawings and accompanying descriptions and examples.

Before explaining at least one embodiment of the invention in detail, it is to be understood that the invention is not limited in its application to the details of construction and the arrangement of the components set forth in the following description or illustrated in the Examples section. The invention is capable of other embodiments or of being practiced or carried out in various ways. Also, it is to be understood that the phraseology and terminology employed herein is for the purpose of description and should not be regarded as limiting.

Organ transplantation is the preferred, indeed the only, effective treatment for many diseases of large organs, such as liver, kidney, heart, etc. However, the technical complexities, exceptionally high costs, risk of disease, almost unavoidable immune complications, and the scarcity of healthy, matched organs for transplantation have made imperative the search for alternative therapeutic methods. Of these, repopulation of organs with transplanted cells seems the most promising.

Numerous studies with small animals have indicated the feasibility of transplantation of cells to repopulate injured or dysfunctional organs. Hepatocytes have been transplanted into host livers with varying degrees of success, the best results achieved with hepatic “oval” cells or fetal liver progenitor cells (see, for example, Sandhu, et al Am. J. Pathol., 2001;159:1323-34, Mahli et al, J. Cell Sci 2002; 115:2679-88 and Wang et al, PNAS USA 2003;100:11881-88). However, hepatocyte transplantation in humans has been to date unsuccessful (Fox et al, Am J Transplant. 2004;56:7-13), with the one successful case of repopulation still needing an organ transplant. The most significant limitation is the availability of large numbers of transplantable cells.

While reducing the present invention to practice, it was found that non-endodermally-derived stem cells can be expanded ex-vivo to provide cells for transplantation into STZ-diabetic hosts, restoring pancreatic function in the diseased host. Thus, according to one aspect of the present invention, there is provided a method of enhancing function of an endodermally derived organ in a subject in need thereof, the method effected by: (a) obtaining a population of cells comprising stem and/or progenitor cells; (b) culturing the stem and/or progenitor cells ex-vivo under conditions allowing for cell proliferation and, at the same time, culturing said cells under conditions selected from the group consisting of: (i) conditions reducing expression and/or activity of CD38 in the cells; (ii) conditions reducing capacity of the cells in responding to signaling pathways involving CD38 in the cells; (iii) conditions reducing capacity of the cells in responding to retinoic acid, retinoids and/or Vitamin D in the cells; (iv) conditions reducing capacity of the cells in responding to signaling pathways involving the retinoic acid receptor, the retinoid X receptor and/or the Vitamin D receptor in the cells; (v) conditions reducing capacity of the cells in responding to signaling pathways involving PI 3-kinase; (vi) conditions wherein the cells are cultured in the presence of nicotinamide, a nicotinamide analog, a nicotinamide or a nicotinamide analog derivative or a nicotinamide or a nicotinamide analog metabolite; (vii) conditions wherein the cells are cultured in the presence of a copper chelator; (viii) conditions wherein the cells are cultured in the presence of a copper chelate; (ix) conditions wherein the cells are cultured in the presence of a PI 3-kinase inhibitor; thereby expanding the stem and/or progenitor cells while at the same time, substantially inhibiting differentiation of the stem and/or progenitor cells ex-vivo; and (c) implanting the cells in an endodermally-derived organ of the subject.

As used herein, the phrase “stem cells” refers to pluripotent cells that, given the right growth conditions, may develop to any cell lineage present in the organism from which they were derived. The phrase, as used herein, refers both to the earliest renewable cell population responsible for generating cell mass in a tissue or body and the very early progenitor cells, which are somewhat more differentiated, yet are not committed and can readily revert to become a part of the earliest renewable cell population. Methods of ex-vivo culturing stem cells of different tissue origins are well known in the art of cell culturing. To this effect, see for example, the text book “Culture of Animal Cells—A Manual of Basic Technique” by Freshney, Wiley-Liss, N. Y. (1994), Third Edition, the teachings of which are hereby incorporated by reference.

As used herein the term “inhibiting” refers to slowing, decreasing, delaying, preventing or abolishing.

As used herein the term “differentiation” refers to relatively generalized or specialized changes during development. Cell differentiation of various lineages is a well-documented process and requires no further description herein. As used herein the term differentiation is distinct from maturation which is a process, although some times associated with cell division, in which a specific cell type mature to function and then dies, e.g., via programmed cell death.

The phrase “cell expansion” is used herein to describe a process of cell proliferation substantially devoid of cell differentiation. Cells that undergo expansion hence maintain their cell renewal properties and are oftentimes referred to herein as renewable cells, e.g., renewable stem cells.

As used herein the term “ex-vivo” refers to a process in which cells are removed from a living organism and are propagated outside the organism (e.g., in a test tube). As used herein, the term “ex-vivo”, however, does not refer to a process by which cells known to propagate only in-vitro, such as various cell lines (e.g., HL-60, MEL, HeLa, etc.) are cultured. In other words, cells expanded ex-vivo according to the present invention do not transform into cell lines in that they eventually undergo differentiation.

Providing the ex-vivo grown cells with conditions for ex-vivo cell proliferation include providing the cells with nutrients and preferably with one or more cytokines, as is further detailed hereinunder.

Ex-vivo expansion of the stem and/or progenitor cells, under conditions substantially inhibiting differentiation, has been described. PCT IL03/00064 to Peled et al, which is incorporated by reference as if fully set forth herein, teaches methods of reducing expression and/or activity of CD38 in cells, methods of reducing capacity of cells in responding to signaling pathways involving CD38 in the cells, methods of reducing capacity of cells in responding to retinoic acid, retinoids and/or Vitamin D in the cells, methods of reducing the capacity of cells in responding to signaling pathways involving the retinoic acid receptor, the retinoid X receptor and/or the Vitanmin D receptor in the cells, methods of reducing the capacity of cells in responding to signaling pathways involving PI 3-kinase, conditions wherein cells are cultured in the presence of nicotinamide, a nicotinamide analog, a nicotinamide or a nicotinamide analog derivative or a nicotinamide or a nicotinamide analog metabolite and conditions wherein cells are cultured in the presence of a PI3-kinase inhibitor.

In one embodiment of the invention, reducing the activity of CD38 is effected by providing the cells with an agent that inhibits CD38 activity (i.e., a CD38 inhibitor).

As used herein a “CD38 inhibitor” refers to an agent which is capable of down-regulating or suppressing CD38 activity in stein cells.

A CD38 inhibitor according to this aspect of the present invention can be a “direct inhibitor” which inhibits CD38 intrinsic activity or an “indirect inhibitor” which inhibits the activity or expression of CD38 signaling components (e.g., the cADPR and ryanodine signaling pathways) or other signaling pathways which are effected by CD38 activity.

According to presently known embodiments of this aspect of the present invention, nicotinamide is a preferred CD38 inhibitor.

Hence, in one embodiment, the method according to this aspect of the present invention is effected by providing the cells either with nicotinamide itself, or with a nicotinamide analog, a nicotinamide or a nicotinamide analog derivative or a nicotinamide or a nicotinamide analog metabolite.

As used herein, the phrase “nicotinamide analog” refers to any molecule that is known to act similarly to nicotinamide. Representative examples of nicotinamide analogs include, without limitation, benzamide, nicotinethioamide (the thiol analog of nicotinamide), nicotinic acid and α-amino-3-indolepropionic acid.

The phrase “a nicotinamide or a nicotinamide analog derivative” refers to any structural derivative of nicotinamide itself or of an analog of nicotinamide. Examples of such derivatives include, without lirnitation, substituted benzamides, substituted nicotinamides and nicotinethioamides and N-substituted nicotinamides and nicotinthioamides.

The phrase “a nicotinamide or a nicotinamide analog metabolite” refers to products that are derived from nicotinamide or from analogs thereof such as, for example, NAD, NADH and NADPH.

Alternatively, a CD38 inhibitor according to this aspect of the present invention can be an activity neutralizing antibody which binds for example to the CD38 catalytic domain, thereby inhibiting CD38 catalytic activity. It will be appreciated, though, that since CD38 is an intracellular protein measures are taken to use inhibitors which may be delivered through the plasma membrane. In this respect a fragmented antibody such as a Fab fragment (described hereinunder) is preferably used.

The term “antibody” as used in this invention includes intact molecules as well as functional fragments thereof, such as Fab, F(ab′)₂, and Fv that are capable of binding to macrophages. These functional antibody fragments are defined as follows:

Fab, the fragment which contains a monovalent antigen-binding fragment of an antibody molecule, can be produced by digestion of whole antibody with the enzyme papain to yield an intact light chain and a portion of one heavy chain;

Fab′, the fragment of an antibody molecule that can be obtained by treating whole antibody with pepsin, followed by reduction, to yield an intact light chain and a portion of the heavy chain; two Fab′ fragments are obtained per antibody molecule;

(Fab′)₂, the fragment of the antibody that can be obtained by treating whole antibody with the enzyme pepsin without subsequent reduction; F(ab′)₂ is a dimer of two Fab′ fragments held together by two disulfide bonds;

Fv, defined as a genetically engineered fragment containing the variable region of the light chain and the variable region of the heavy chain expressed as two chains; and

Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

Antibody fragments according to the present invention can be prepared by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, furter cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Natl Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins recipient antibody in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Alternatively, the method according to this aspect of the present invention can be effected by providing the ex-vivo cultured stem cells with an agent that down-regulates CD38 expression.

An agent that downregulates CD38 expression refers to any agent which affects CD38 synthesis (decelerates) or degradation (accelerates) either at the level of the mRNA or at the level of the protein. For example, a small interfering polynucleotide molecule which is designed to down regulate the expression of CD38 can be used according to this aspect of the present invention.

An example of a small interfering polynucleotide molecule which can down-regulate the expression of CD38 is a small interfering RNA or siRNA, such as, for example, the morpholino antisense oligonucleotides described by in Munshi et al. (Munshi C B, Graeff R, Lee H C, J Biol Chem Dec. 20, 2002;277(51):49453-8), which includes duplex oligonucleotides which direct sequence specific degradation of mRNA through the previously described mechanism of RNA interference (RNAi) (Hutvagner and Zamore (2002) Curr. Opin. Genetics and Development 12:225-232).

As used herein, the phrase “duplex oligonucleotide” refers to an oligonucleotide structure or mimetics thereof, which is formed by either a single self-complementary nucleic acid strand or by at least two complementary nucleic acid strands. The “duplex oligonucleotide” of the present invention can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA.

Preferably, the specific small interfering duplex oligonucleotide of the present invention is an oligoribonucleotide composed mainly of ribonucleic acids.

Instructions for generation of duplex oligonucleotides capable of mediating RNA interference are provided in www.ambion.com.

Hence, the small interfering polynucleotide molecule according to the present invention can be an RNAi molecule (RNA interference molecule).

Alternatively, a small interfering polynucleotide molecule can be an oligonucleotide such as a CD38-specific antisense molecule or a rybozyme molecule, further described hereinunder.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this embodiment of the present invention are those having a length selected from a range of 10 to about 200 bases preferably 15-150 bases, more preferably 20-100 bases, most preferably 20-50 bases.

The oligonucleotides of the present invention may comprise heterocyclic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360;

5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374, which is herein incorporated by reference. Oligonucleotides of the present invention may also include base modifications or substitutions. As used herein, “unmodified” or “natural” bases include the purine bases adenine (A) and guanine (G), and the pyrimidine bases thymine (I), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

As described hereinabove, the oligonucleotides of the present invention are preferably antisense molecules, which are chimeric molecules. “Chimeric antisense molecules” are oligonucleotides, which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids. An example for such includes RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

The oligonucleotides of the present invention can flrther comprise a ribozyme sequence. Ribozymes are being increasingly used for the sequence-specific inhibition of gene expression by the cleavage of mRNAs. Several rybozyme sequences can be fused to the oligonucleotides of the present invention. These sequences include but are not limited ANGIOZYME specifically inhibiting formation of the VEGF-R (Vascular Endothelial Growth Factor receptor), a key component in the angiogenesis pathway, and HEPTAZYME, a rybozyme designed to selectively destroy Hepatitis C Virus (HCV) RNA, (Rybozyme Pharmaceuticals, Incorporated—WEB home page).

Further alternatively, a small interfering polynucleotide molecule, according to the present invention can be a DNAzyme.

DNAzymes are single-stranded catalytic nucleic acid molecules. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M Curr Opin Mol Ther 2002;4:119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Alternatively, as described hereinabove, retinoid receptor superfamily inhibitors (e.g., antagonists, siRNA molecules, antisense molecules, antibodies, etc.) which downregulate or suppress retinoid receptor activity and/or expression can be used to down regulate CD38 expression.

Briefly, retinoid receptors such as RAR, RXR and VDR have been reported to be involved in the regulation of gene expression pathways associated with cell proliferation and differentiation and in particular in the regulation of CD38 expression. Hence, preferred agents that downregulate CD38 expression according to the present invention include RAR antagonists, RXR antagonists and VDR antagonists or, alternatively, antagonists for reducing the capacity of the stem cells in responding to retinoic acid, retinoid and/or Vitamin D.

As used herein the term “antagonist” refers to an agent that counteracts or abrogates the effects of an agonist or a natural ligand of a receptor. Further features relating to such antagonists are detailed hereinunder.

In one preferred embodiment, reducing the capacity of the stem cells in responding to the above antagonists and/or signaling pathways of the above receptors and kinase is by ex-vivo culturing the stem cells in a presence of an effective amount of at least one retinoic acid receptor antagonist, at least one retinoid X receptor antagonist and/or at least one Vitamin D receptor antagonist, preferably, for a time period of 0.1-50%, preferably, 0.1-25%, more preferably, 0.1-15%, of an entire ex-vivo culturing period of the stem cells or for the entire period. In this respect it was surprisingly uncovered that an initial pulse exposure to an antagonist is sufficient to exert cell expansion long after the antagonist was removed from the culturing set up.

Many antagonists to RAR, RXR and VDR are presently known, some of which are listed hereinafter.

The retinoic acid receptor antagonist used in context of the different aspects and embodiments of the present invention can be: AGN 194310; AGN 109; 3-4-Methoxy-phenylsulfanyl)-3-methyl-butyric acid; 6-Methoxy-2,2-dimethvl-thiochroman-4-one,2,2-Dimethyl-4-oxo-thiochroman-6-yltrifluoromethane-sulfonate; Ethyl 4-((2,2 dimethyl-4-oxo-thiochroman-6-yl)ethynyl)-benzoate; Ethyl 4-((2,2-dimethy 1-4-triflouromethanensulfonyloxy-(2H)-thiochromen-6-yl)ethynyl)-benzoate(41); Thiochromen-6-yl]-ethynyl]-benzoate(yl); (p-[(E)-2-(3′4′-Dihydro-4,4′-dimethyl-7′-heptyloxy)-2′H-1-benzothiopyran-6′yl]propenyl]benzoic acid 1′1′-dioxide; 2E,4E,6E-[7-(3,5-Di-t-butyl-4-n-butoxyphenyl)-3-methyl]-octa-2,4,6-trienoic acid; 2E,4E,6E-[7-(3,5-Di-t-butyl-4-n-propoxyphenyl)-3-methyl]-cta-2,4,6-trienoic acid; 2E,4E,6E-[7-(3,5-Di-t-butyl-4-n-pentoxyphenyl)-3-methyl]-octa-2,4,6-trienoic acid; 2E,4E,6E-[7-(3,5-Di-t-butyl-4-n-hexoxyphenyl)-3-methyl]-octa-2,4,6-trienoic acid; 2E,4E,6E-[7-(3,5-Di-t-butyl-4-n-heptoxyphenyl)-3-methyl]-octa-2,4,6-trienoic acid; 2E,4E,6E-[7-(3,5-Di-t-butyl-4-n-octoxyphenyl)-3-methyl]-octa-2,4,6-trienoic acid; (2E,4E,6E)-7-[3-t-butyl-5-(1-phenyl-vinyl)-phenyl]-3-methyl-octa-2,4,6-trienoic acid; 2E,4E,6E-[7-(3,5-Di-t-butyl-4-{[4,5-.sup.3 H.sub.2]-n-pentoxy}phenyl)-3-methyl]-octa-2,4,6-trienoic acid; (2E,4E)-(1RS,2RS)-5-[2-(3,5-di-tert.butyl-2-ethoxy-phenyl)-cyclopropyl]-3-methyl-penta-2,4-dienoic acid ethyl ester; (2E,4E)-(1RS,2RS)-5-[2-(3,5-di-tert.butyl-2-ethoxy-phenyl)-cyclopropyl]-3-methyl-penta-2,4dienoic acid; (2E,4E)-(1RS,2RS)-5-[2-(3,5-di-tert.butyl-2-butoxy-phenyl)-cyclopropyl]-3-methyl-penta-2,4-dienoic acid; (2E,4E,6Z)-7-[3,5-di-tert.butyl-2-ethoxyphenyl]3-methyl-2,4,6-octatrienoic acid; (2E,4E,6Z)-7-[3,5-di-tert.butyl-2-butyloxyphenyl]-3-methyl-2,4,6-octatrienoic acid; 4-(5,6,7,8-tetrahydro-5,5,8,8-tetramethyl-2-naphthalene-carboxamido)benzoic acid; (2E,4E)-3-methyl-5-[(1S,2S)-2-(5,5,8,8-tetramethyl-5,6,7,8-tetrahydro-naphthalen-2-yl)-cyclopropyl]-penta-2,4-dienoic acid; p-[(E)-2-[3′,4′-Dihydro-4′,4′-dimethyl-7′-(heptyloxy)-2′H-1-benzothiopyran-6′-yl]propenyl]benzoic acid; 1′,1′-dioxide, 4-(7,7,10,10-Tetramethyl-1-pyridin-3-ylmethyl-4,5,7,8,9,10-hexahydro-1H-naphto[2,3-g]indol-3-yl)-benzoic acid; (2E,4E,6Z)-7-[3,5-di-tert.butyl-2-methoxyphenyl]-3-methyl-2,4,6-octatrienoic acid; (2E,4E,6Z)-7-[3,5-di-tert.butyl-2-ethoxyphenyl]-3-methyl-2,4,6-octatrienoic acid; (2E,4E,6Z)-7-[3,5-di-tert.butyl-2-hexyloxyphenyl]-3-methyl-2,4,6-octatrienoic acid; (2E,4E,6Z)-7-[3,5-di-tert.butyl-2-octyloxyphenyl]-3-methyl-2,4,6-octatrienoic acid; and (2E,4E)-(1RS,2RS)-5-[2-(3,5-di-tert-butyl-2-butoxy-phenyl)-cyclopropyl]-3-methyl-penta-2,4-dienoic acid (2E,4E,6Z)-7-(3-n-propoxy-5,6,7,8-tetrahydro-5,5,8,8-tetramethylnaphthalene-2-yl)-3-methylocta-2,4,6-trienoic acid, and4-(5H-2,3(2,5 dimethyl-2,5-hexano)-5-n-propyldibenzo[b,e]][1,4]diazepin-11-yl)benzoic acid, and 4-(5H-2,3-(2,5-dimethyl-2,5-hexano)-5methyl-8-nitrodibenzo[b,e][1,4]diazepin-11-yl)benzoic acid, and 4-{[4-(4-Ethylphenyl)2,2-dimethyl-(2H)-thiochromen-6-yl]ethynyl}benzoic acid, and 4-[4-2methyl-1,2-dicarba-closo-dodecaboran-1-yl-phenylcarbamoyl]benzoic acid, and4-[4,5,7,8,9,10-hexahydro-7,7,10,10-tetramethyl-1-(3-pyridylmethyl)-anthra[1,2-b]pyrrol-3-yl]benzoic acid, and (3-pyridylmethyl)-]5-thiaanthra[2,1-b]pyrrol-3-yl)benzoic acid, and (3-pyridylmethyl)-anthra[2ml-d]pyrazol-3-yl]benzoic acid.

The retinoid X receptor antagonist used in context of the different aspects and embodiments of the present invention can be: LGN100572, 1-(3-hydroxy-5,6,7,8-tetrahydro-5,5,8,8-tetramethylnaphthalene-2-yl)ethanone, 1-(3-propoxy-5,6,7,8-tetrahydro-5,5,8,8-tetramethylnaphthalene-2-yl)ethanone, 3-(3-propoxy-5,6,7,8-tetrahydro-5,5,8,8-tetramethylnaphthalene-2-yl)but-2-enenitrile, 3-(3-propoxy-5,6,7,8-tetrahydro-5,5,8,8-tetramethylnaphthalene-2-yl)but-2-enal, (2E,4E,6E)-7-3[-propoxy-5,6,7,8-tetrahydro 5,5,8,8-tetramethyl-2-naphthalene-2-yl]-3-methylocta-2,4,6-trienoic acid, 4-[3,5,5,8,8-pentamethyl-5,6,7,8-tetrahydro-2-naphthyl)carbonyl]benzoic acid, 4-[1-(3,5,5,8,8-pentamethyl-5,6,7,8-tetrahydro-2-naphthyl)ethenyl]benzoic acid, 4-[1(3,5,5,8,8-pentamethyl-5,6,7,8-tetrahydro-2-naphthyl)cyclopropyl]benzoic acid, 4-[1-(3,5,5,8,8-pentamethyl-5,6,7,8-tetrahydro-2-naphthyl)ethenyl]benzenete trazole, 2-[1-(5,5,8,8-tetramethyl-5,6,7,8-tetrahydro-2-naphthyl)ethenyl]pyridine-5-carboxylic acid, 2-[1-(3,5,5,8,8-pentamethyl-5,6,7,8-tetrahydro-2-naphthyl)ethyl]pyridine-5-carboxylic acid, ethyl-2-[1-(3,5,5,8, 8-pentamethyl-5,6,7,8-tetrahydro-2-naphthyl)ethenyl]pyridine-5-carboxylate, 5-[1-3,5,5,8,8-pentamethyl-5,6,7,8-tetrahydro-2-naphthyl)ethenyl]pyridine-2-carboxylic acid, 2-[1-(3,5,5,8,8-pentamethyl-5,6,7,8-tetrahydro-2-naphthyl)cyclopropyl]pyridine-5-carboxylic acid, methyl 2-[1-(3,5,5,8,8-pentamethyl-5,6,7,8-tetrahydro-2-naphthyl)cyclopropyl]pyridine-5-carboxylate, 4-[1-(3,5,5,8,8-pentamethyl-5,6,7,8-tetrahydro-2-naphthyl)ethenyl]-N-(4-hydroxyphenyl)benzamide, 2-[1-(3,5,5,8,8-Pentamethyl-5,6,7,8-tetrahydro-2-naphthyl)ethenyl]pyridine-5-carboxylic acid, 2-[1-(3,5,5,8,8-Pentamethyl-5,6,7,8-tetrahydro-2-naphthyl)cyclopropyl]pyridine-5-carboxylic acid, 4-[(3,5,5,8,8-pentamethyl-5,6,7,8-tetrahydro-2-naphthyl)carbonyl]benzoic acid butyloxime, 4-[(3,5,5,8,8-pentamethyl-5,6,7,8-tetrahydro-2-naphthyl)carbonyl]benzoic acid propyloxime, 4-[(3,5,5,8,8-pentamethyl-5,6,7,8-tetrahydro-2-naphthyl)carbonyl]benzoic acid cyanoimine, 4-[(3,5,5,8,8-pentamethyl-5,6,7,8-tetrahydro-2-naphthyl)carbonyl]benzoic acid allyloxime, 4-[(3,5,5,8,8-pentamethyl-5,6,7,8-tetrahydro-2-naphthyl)carbonyl]benzoic acid 4-(3-methylbut-2-enoic acid)oxime, and 4-[(3,5,5,8,8-pentamethyl-5,6,7,8-tetrahydro-2-naphthyl)carbonyl]benzoic acid 1-aminoethyloxime (2E,4E,6Z)-7-(3-n-propoxy-5,6,7,8-tetrahydro-5,5,8,8-tetramethylnaphthalene-2-yl)-3-methylocta-2,4,6-trienoic acid, and4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5m.

The Vitamin D receptor antagonist used in context of the different aspects and embodiments of the present invention can be: I alpha, 25-(OH)-D3-26,23 lactone; 1 alpha, 25-dihydroxyvitamin D (3); the 25-carboxylic ester ZK159222; (23S)-25-dehydro-1 alpha-OH-D (3); (23R)-25-dehydro-1 alpha-OH-D (3);. 1 beta, 25 (OH)₂ D₃; 1 beta, 25(OH)₂-3-epi-D₃; (23S) 25-dehydro-1 alpha(OH) D3-26,23-lactone; (23R) 25-dehydro-1 alpha(OH)D3-26,23-lactone and Butyl-(5Z,7E,22E-(1S,7E,22E-(1S,3R,24R)-1,3,24-trihydroxy-26,27-cyclo-9,10-secocholesta-5,7,10(19),22-tetraene-25-carboxylate).

The above listed antagonists are known for their high affinity towards their respective cognate receptors. However, it may be possible for these molecules to be active towards other receptors.

Each of the agents described hereinabove may reduce the expression or activity of CD38 individually. However, the present invention aims to also encompass the use of any subcombination of these agents.

It will be appreciated that protein agents (e.g., antibodies) of the present invention can be expressed from a polynucleotide encoding same and provided to ex-vivo cultured stem cells employing an appropriate gene delivery vehicle/method and a nucleic acid construct as is further described hereinunder.

Examples of suitable constructs include, but are not limited to pcDNA3, pcDNA3.1 (±), pGL3, PzeoSV2 (±), pDisplay, pEP/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

As the method of ex-vivo expanding a population of stem cells, while at the same time, substantially inhibiting differentiation of the stem cells ex-vivo, according to this aspect of the present invention, is effected by modulating CD38 expression and/or activity, either at the protein level, using RAR, RXR or VDR antagonists or a CD38 inhibitor such as nicotinamide and analogs thereof, or at the at the expression level via genetic engineering techniques, as is detailed hereinabove, there are further provided, according to the present invention, several preferred methods of ex-vivo expanding a population of stem cells, while at the same time, substantially inhibiting differentiation of the stem cells ex-vivo.

Still alternatively, according to the present invention, as described hereinabove, inhibitors of activity or expression of PI 3-kinase are used to down regulate CD38 expression.

It will be appreciated, in the context of the present invention, that Hori et al (PNAS USA 2002;99:16105-10) reported that treatment of mouse embryonic stem cells with inhibitors of phosphoinositide 3-kinase caused differentiation of the stem cells, producing cells that resembled pancreatic β cells, which were implanted into diabetic mice for restoration of pancreas function. Thus, the prior art teaches away from the methods of the present invention.

In stark contrast, PCT IL2004/000215 to Peled et al., which is incorporated by reference as if fully set forth herein, discloses the use of inhibitors of PI 3-K activity or expression for ex-vivo expansion of stem and/or progenitor cells while inhibiting differentiation thereof.

Thus, in still another particular embodiment of this aspect of the present invention, culturing the stem and/or progenitor cells ex-vivo under conditions allowing for cell proliferation and at the same time inhibiting differentiation is effected by culturing the cells in conditions reducing the capacity of the cells in responding to signaling pathways involving PI 3-kinase, or in conditions wherein the cells are cultured in the presence of the PI 3-kinase inhibitors.

All the methodologies described herein with respect to the inhibition of expression apply also to inhibition of expression of PI 3-kinase. These methodologies include, for example, the use of polynucleotides, such as small interfering RNA molecules, antisense ribozymes and DNAzymes, as well as intracellular antibodies.

Inhibition of PI 3-kinase activity can be effected by known PI 3-kinase inhibitors, such as wortmannin and LY294002 and the inhibitors described in, for example, U.S. Pat. No. 5,378,725, which is incorporated herein by reference. In one particular embodiment, the ex-vivo expanding a population of stem cells, while at the same time, substantially inhibiting differentiation of the stem cells ex-vivo is effected by providing the stem cells with ex-vivo culture conditions for ex-vivo cell proliferation and, at the same time, for reducing a capacity of the stem cells in responding to retinoic acid, retinoids and/or Vitamin D, thereby expanding the population of stem cells while at the same time, substantially inhibiting differentiation of the stem cells ex-vivo. In still another particular embodiment of this aspect of the present invention, the ex-vivo expanding a population of stem cells, while at the same time, substantially inhibiting differentiation of the stem cells ex-vivo is effected by obtaining adult or neonatal umbilical cord whole white blood cells or whole bone marrow cells sample and providing the cells in the sample with ex-vivo culture conditions for stem cells ex-vivo cell proliferation and with a PI 3-kinase inhibitor, thereby expanding a population of a renewable stem cells in the sample.

In one preferred embodiment, concomitant with treating the cells with conditions which allow for ex-vivo the stem cells to proliferate, the cells are short-term treated or long-term treated to reduce the expression and/or activity of PI 3-kinase.

In one embodiment of the invention, reducing the activity of PI 3-kinase is effected by providing the cells with an modulator of PI 3-kinase that inhibits PI 3-kinase catalytic activity (i.e., a PI 3-kinase inhibitor).

As used herein a “modulator capable of downregulating PI 3-kinase activity or gene expression” refers to an agent which is capable of down-regulating or suppressing PI 3-kinase activity in stem cells.

An inhibitor of PI 3-kinase activity according to this aspect of the present invention can be a “direct inhibitor” which inhibits PI 3-kinase intrinsic activity or an “indirect inhibitor” which inhibits the activity or expression of PI 3-kinase signaling components (e.g., the Akt and PDK1 signaling pathways) or other signaling pathways which are effected by PI 3-kinase activity.

According to presently known embodiments of this aspect of the present invention, wortmannin and LY294002 are preferred PI 3-kinase inhibitors.

Hence, in one embodiment, the method according to this aspect of the present invention is effected by providing known PI 3-kinase inhibitors, such as wortmannin, LY294002, and active derivatives thereof, as described in, for example, U.S. Pat. Nos. 5,378,725, 5,480,906, 5,504,103, and in International Patent Publications WO 03072557, and WO 9601108, all of which are incorporated herein by reference, and by the specific PI 3-kinase inhibitors disclosed in US Patent Publication 20030149074 to Melese et al., also incorporated herein by reference.

Phosphatidylinositol 3-kinase inhibitors are well known to those of skill in the art. Such inhibitors include, but are not limited to Ly294002 (Calbiochem Corp., La Jolla, Calif.) and wortmannin (Sigma Chemical Co., St. Louis Mo.) which are both potent and specific PI3K inhibitors. The chemical properties of Ly294002 are described in detail in J. Biol., Chem., (1994) 269: 5241-5248. Briefly, Ly294002, the quercetin derivative, was shown to inhibit phosphatidylinositol 3-kinase inhibitor by competing for phosphatidylinositol 3-kinase binding of ATP. At concentrations at which LY294002 fully inhibits the ATP-binding site of PI3K, it has no inhibitory effect against a number of other ATP-requiring enzymes including PI4-kinase, EGF receptor tyrosine kinase, src-like kinases, MAP kinase, protein kinase A, protein kinase C, and ATPase.

LY294002 is very stable in tissue culture medium, is membrane permeable, has no significant cytotoxicity, and at concentrations at which it inhibits members of PI3K family, it has no effect on other signaling molecules.

Phosphatidylinositol 3-kinase, has been found to phosphorylate the 3-position of the inositol ring of phosphatidylinositol (PI) to form phosphatidylinositol 3-phosphate (PI-3P) (Whitman et al.(1988) Nature, 322: 664-646). In addition to PI, this enzyme also can phosphorylate phosphatidylinositol 4-phosphate and phosphatidylinositol 4,5-bisphosphate to produce phosphatidylinositol 3,4-bisphosphate and phosphatidylinositol 3,4,5-trisphosphate (PIP3), respectively (Auger et al. (1989) Cell, 57: 167-175). PI 3-kinase inhibitors are materials that reduce or eliminate either or both of these activities of PI 3-kinase. Identification, isolation and synthesis of such inhibitors is disclosed in U.S. Pat. No. 6,413,773 to Ptasznik et al.

The phrase “active derivative” refers to any structural derivative of wortmannin or LY294002 having a PI 3-kinase downregulatory activity, as measured, for example, by catalytic activity, binding studies, etc, in vivo or in vitro.

Alternatively, a modulator downregulating PI 3-kinase activity or gene expression according to this aspect of the present invention can be an activity neutralizing anti-PI 3-kinase antibody which binds, for example to the PI 3-kinase catalytic domain, or substrate binging site, thereby inhibiting PI 3-kinase catalytic activity. It will be appreciated, though, that since PI 3-kinase is an intracellular protein measures are taken to use modulators which may be delivered through the plasma membrane. In this respect a fragmented antibody such as a Fab fragment (described hereinunder), or a genetically engineered ScFv is preferably used.

A modulator that downregulates PI 3-kinase expression refers to any agent which affects PI 3-kinase synthesis (decelerates) or degradation (accelerates) either at the level of the mRNA or at the level of the protein. For example, downregulation of PI 3-kinase expression can be achieved using oligonucleotide molecules designed to specifically block the transcription of PI 3-kinase mRNA, or the translation of PI 3-kinase transcripts at the ribosome, can be used according to this aspect of the present invention. In one embodiment, such oligonucleotides are antisense oligonucleotides.

Design of antisense molecules which can be used to efficiently inhibit PI 3-kinase expression must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof. Sequences suitable for use in construction and synthesis of oligonucleotides which specifically bind to PI 3-kinase mRNA, genomic DNA, promoter and/or other control sequences of PI 3-kinase are available in published PI 3-kinase nucleotide sequences, including, but not limited to, GenBank Accession Nos: AF327656 (human gamma catalytic subunit); NM006219 (human beta subunit); NM002647 (human class III); NM181524 (human p85 alpha subunit); U86453 (human p110 delta isoform); and S67334 (human p110 beta isoform).

The prior art teaches of a number of delivery strategies which can be used to efficiently deliver oligonucleotides into a wide variety of cell types (see, for example, Luft (1998) J Mol Med 76(2): 75-6; Kronenwett et al. (1998) Blood 91(3): 852-62; Rajur et al. (1997) Bioconjug Chem 8(6): 935-40; Lavigne et al. (1997) Biochem Biophys Res Commun 237(3): 566-71 and Aoki et al. (1997) Biochem Biophys Res Commun 231(3): 540-5).

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. (1999) Biotechnol Bioeng 65(1): 1-9].

Such algorithms have been successfully employed to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al. (1998) Nature Biotechnology 16, 1374-1375). Examples of antisense molecules which have been demonstrated capable of down-regulating the expression of PI 3-kinase are the PI 3-kinase specific antisense oligonucleotides described by Mood et al (Cell Signal 2004;16:631-42), incorporated herein by reference. The production of PI 3-kinase-specific antisense molecules is disclosed by Ptasznik et al (U.S. Pat. No. 6,413,773), incorporated herein by reference.

Reducing the capacity of the cells in responding to retinoic acid, retinoids and/or Vitamin D, or to retinoic acid, retinoid X and/or Vitamin D receptor signaling may be effected, for example, by the administration of chemical inhibitors, including receptor antagonists. In another particular, the method of ex-vivo expanding a population of stem cells, while at the same time, substantially inhibiting differentiation of the stem cells ex-vivo is effected by providing the stem cells with ex-vivo culture conditions for ex-vivo cell proliferation and, at the same time, for reducing a capacity of the stem cells in responding to signaling pathways involving the retinoic acid receptor, retinoid-X receptor and/or Vitamin D receptor, thereby expanding the population of stem cells while at the same time, substantially inhibiting differentiation of the stem cells ex-vivo. Reducing the capacity of the cells to respond to retinoic acid, retinoid X and/or Vitamin D receptor signaling events, includes treating the cells with antagonists supplied continuously or for a short-pulse period, and is effected by a diminution or abrogation of cellular signaling pathways through their respective, cognate receptors.

Final concentrations of the antagonists may be, depending on the specific application, in the micromolar or millimolar ranges. For example, within about 0.1 μM to about 100 mM, preferably within about 4 μM to about 50 mM, more preferably within about 5 μM to about 40 mM.

Final concentrations of the nicotinamide or the analogs, derivatives or metabolites thereof and of the PI 3-kinase inhibitor are preferably, depending on the specific application, in the millimolar ranges. For example, within about 0.1 mM to about 20 mM, preferably within about 1 mM to about 10 mM, more preferably within about 5 mM to about 10 mM.

In still another particular embodiment of this aspect of the present invention, culturing the stem and/or progenitor cells ex-vivo under conditions allowing for cell proliferation and at the same time inhibiting differentiation is effected by culturing the cells in the presence of a copper chelator. PCT IL99/00444 to Peled, et al, which is incorporated by reference as if fully set for herein, discloses the use of heavy metal chelators, having high affinity for copper, for efficient ex-vivo expansion of stem and/or progenitor cells, while substantially inhibiting differentiation thereof.

Final concentrations of the chelator may be, depending on the specific application, in the micromolar or millimolar ranges. For example, within about 0.1 μM to about 100 mM, preferably within about 4 μM to about 50 mM, more preferably within about 5 μM to about 40 mM.

According to a preferred embodiment of the invention the chelator is a polyamine chelating agent, such as, but not limited to ethylendiamine, diethylenetriamine, triethylenetetramine, triethylenediamine, tetraethylenepentamine, aminoethylethanolamine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine, N,N′-bis(3-aminopropyl)-1,3-propanediamine, N,N,Bis(2 animoethyl)1,3 propane diamine, 1,7-dioxa-4,10-diazacyclododecane, 1,4,8,11-tetraaza cyclotetradecane-5,7-dione, 1,4,7-triazacyclononane trihydrochloride, 1-oxa-4,7,10-triazacyclododecane, 1,4,8,12-tetraaza cyclopentadecane or 1,4,7,10-tetraaza cyclododecane, preferably tetraethylpentamine. The above listed chelators are known in their high affinity towards Copper ions.

In yet another particular embodiment of this aspect of the present invention, culturing the stem and/or progenitor cells ex-vivo under conditions allowing for cell proliferation and at the same time inhibiting differentiation is effected by culturing the cells in the presence of a copper chelate. PCT IL03/00062 to Peled, et al, which is incorporated by reference as if fully set for herein, discloses the use of copper chelates, complexes of copper and heavy metal chelators having high affinity for copper, for efficient ex-vivo expansion of stem and/or progenitor cells, while substantially inhibiting differentiation thereof.

The copper chelate, according to the present invention, is used in these and other aspects of the present invention, in the context of expanding a population of stem and/or progenitor cells, while at the same time reversibly inhibiting differentiation of the stem and/or progenitor cells. Providing the cells with the copper chelate maintains the free copper concentration available to the cells substantially unchanged.

The copper chelate according to the present invention is oftentimes capable of forming an organometallic complex with a transition metal other than copper. As metals other than copper are typically present in the cells (e.g., zinc) or can be administered to cells during therapy (e.g., platinum), it was found that copper chelates that can also interact with other metals are highly effective. Representative examples of such transition metals include, without limitation, zinc, cobalt, nickel, iron, palladium, platinum, rhodium and ruthenium.

The copper chelates of the present invention comprise copper ion (e.g., Cu⁺¹, Cu⁺²) and one or more chelator(s). As is discussed hereinabove, preferred copper chelators include polyamine molecules, which can form a cyclic complex with the copper ion via two or more amine groups present in the polyamine.

Hence, the copper chelate used in the context of the different aspects and embodiments of the present invention preferably includes a polyamine chelator, namely a polymeric chain that is substituted and/or interrupted with 1-10 amine moieties, preferably 2-8 amine moieties, more preferably 4-6 amine moieties and most preferably 4 amine moieties.

The phrases “amine moiety”, “amine group” and simply “amine” are used herein to describe a —NR′R″ group or a —NR′— group, depending on its location within the molecule, where R′ and R″ are each independently hydrogen, alkyl, cycloalkyl, aryl, heteroaryl or heterocyclic, as these terms are defined hereinbelow.

The polyamine chelator can be a linear polyamine, a cyclic polyamine or a combination thereof.

A linear polyamine, according to the present invention, can be a polyamine that has a general formula I:
HX-Am-(Y₁B₁)₁ . . . (YnBn)n-ZH Formula I
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; and B₁ and Bn are each independently an alkylene chain having between 1 and 20 substituted and/or non-substituted carbon atoms, provided that at least one of X, Z, Y, and Yn is a —NH group and/or at least one of the carbon atoms in the alkylene chains is substituted by an amine group.

Hence, the linear polyamine, according to the present invention, is preferably comprised of one or more alkylene chains (Am, B₁ . . . Bn, in Formula I), is interrupted by one or more heteroatoms such as S, O and N (Y₁ . . . Yn in Formula I), and terminates with two such heteroatoms (X and Z in Formula I).

Alkylene chain A, as is described hereinabove, includes 1-10 substituted or non-substituted carbon atoms and is connected, at least at one end thereof, to a heteroatom (e.g., X in Formula 1). Whenever there are more than one alkylene chains A (in cases where m is greater than one), only the first alkylene chain A is connected to X. However, m is preferably 1 and hence the linear polyamine depicted in Formula I preferably includes only one alkylene chain A.

Alkylene chain B, as is described hereinabove, includes between 1 and 20 substituted or non-substituted carbon atoms. The alkylene chain B is connected at its two ends to a heteroatom (Y₁ . . . Yn and Z in Formula I).

The preferred linear polyamine delineated in Formula I comprises between 1 and 20 alkylene chains B, denoted as B₁ . . . Bn, where “B₁ . . . Bn” is used herein to describe a plurality of alkylene chains B, namely, B₁, B₂, B₃, . . . , Bn-1 and Bn, where n equals 0-20. These alkylene chains can be the same or different. Each of B₁ . . . Bn is connected to the respective heteroatom Y₁ . . . Yn, and the last alkylene chain in the structure, Bn, is also connected to the heteroatom Z.

It should be noted that herein throughout, whenever an integer equals 0 or whenever a component of a formula is followed by the digit 0, this component is absent from the structure. For example, if n in Formula I equals 0, there is no alkylene chain B and no heteroatom Y are meant to be in the structure.

Preferably, n equals 2-10, more preferably 2-8 and most preferably 3-5. Hence, the linear polyamine depicted in Formula I preferably includes between 3 and 5 alkylene chains B, each connected to 3-5 heteroatoms Y.

The linear polyamine depicted in Formula I must include at least one amine group, as this term is defined hereinabove, preferably at least two amine groups and more preferably at least four amine groups. The amine group can be present in the structure as the heteroatoms X, Z or Y₁ . . . Yn, such that at least one of X, Z and Y₁ . . . Yn is a —NH— group, or as a substituent of one or more of the substituted carbon atoms in the alkylene chains A and B₁ . . . Bn. The presence of these amine groups is required in order to form a stable chelate with the copper ion, as is discussed hereinabove.

The alkylene chain A preferably has a general Formula II: 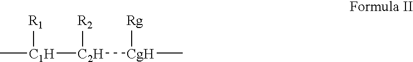
wherein g is an integer that equals 0 or 3-10.

Hence, the alkylene chain A is comprised of a plurality of carbon atoms C₁, C₂, C₃ . . . , Cg−1 and Cg, substituted by the respective R₁, R₂, R₃ . . . , Rg−1 and Rg groups. Preferably, the alkylene chain A includes 2-10 carbon atoms, more preferably, 2-6 and most preferably 2-4 carbon atoms.

As is defined hereinabove, in cases where g equals 0, the component CgH(Rg) is absent from the structure and hence the alkylene chain A comprises only 2 carbon atoms.

R₁, R₂ and Rg are each a substituent attached to the carbon atoms in A. Each of R₁, R₂ and Rg can independently be a substituent such as, but not limited to, hydrogen, alkyl, alkenyl, alkynyl, aryl, cycloalkyl, heteroalicyclic, heteroaryl, halo, amino, alkylamino, arylamino, cycloalkylamino, heteroalicyclic amino, heteroarylamino, hydroxy, alkoxy, aryloxy, azo, C-amido, N-amido, ammonium, thiohydroxy, thioalkoxy, thioaryloxy, sulfonyl, sulfinyl, N-sulfonamide, S-sulfonamide, phosphonyl, phosphinyl, phosphonium, carbonyl, thiocarbonyl, C-carboxy, O-carboxy, C-thiocarboxy, O-thiocarboxy, N-carbamate, O-carbamate, N-thiocarbamate, O-thiocarbamate, urea, thiourea, borate, borane, boroaza, silyl, siloxy, silaza, aquo, alcohol, peroxo, amine oxide, hydrazine, alkyl hydrazine, aryl hydrazine, nitric oxide, cyanate, thiocyanate, isocyanate, isothiocyanate, cyano, alkylnitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, carboxylic acid, alkyl carboxylic acid, aryl carboxylic acid, sulfate, sulfite, bisulfite, thiosulfate, thiosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, phosphate, thiophosphate, phosphite, pyrophosphite, triphosphate, hydrogen phosphate, dihydrogen phosphate, guanidino, S-dithiocarbamate, N-dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraarylborate, tetraalkyl borate, tartrate, salicylate, succinate, citrate, ascorbate, saccharirate, amino acid, hydroxamic acid and thiotosylate.

Whenever R₁, R₂ or Rg is hydrogen, its respective carbon atom in a non-substituted carbon atom.

As used herein, the term “alkyl” is a saturated aliphatic hydrocarbon including straight chain and branched chain groups. Preferably, the alkyl group has 1 to 20 carbon atoms. More preferably, it is a medium size alkyl having 1 to 10 carbon atoms. Most preferably, it is a lower alkyl having 1 to 4 carbon atoms. The alkyl group may be substituted or non-substituted. When substituted, the substituent group can be, for example, cycloalkyl, aryl, heteroaryl, heteroalicyclic, hydroxy, alkoxy, aryloxy, thiohydroxy, thioalkoxy, thioaryloxy, cyano, halo, carbonyl, thiocarbonyl, O-carbamate, N-carbamate, O-thiocarbamate, N-thiocarbamate, C-amido, N-amido, C-carboxy, O-carboxy, nitro, sulfonamide, silyl, guanidine, urea or amino, as these terms are defined hereinbelow.

The term “alkenyl” describes an alkyl group which consists of at least two carbon atoms and at least one carbon-carbon double bond.

The term “alkynyl” describes an alkyl group which consists of at least two carbon atoms and at least one carbon-carbon triple bond.

The term “cycloalkyl” describes an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, aryl, heteroaryl, heteroalicyclic, hydroxy, alkoxy, aryloxy, thiohydroxy, thioalkoxy, thioaryloxy, cyano, halo, carbonyl, thiocarbonyl, C-carboxy, O-carboxy, O-carbamate, N-carbamate, C-amido, N-amido, nitro, or amino, as these terms are defined hereinabove or hereinbelow.

The term “aryl” describes an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, halo, trihalomethyl, alkyl, hydroxy, alkoxy, aryloxy, thiohydroxy, thiocarbonyl, C-carboxy, O-carboxy, O-carbamate, N-carbamate, O-thiocarbamate, N-thiocarbamate, C-amido, N-amido, sulfinyl, sulfonyl or amino, as these terms are defined hereinabove or hereinbelow.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, halo, trihalomethyl, hydroxy, alkoxy, aryloxy, thiohydroxy, thiocarbonyl, sulfonamide, C-carboxy, O-carboxy, sulfinyl, sulfonyl, O-carbamate, N-carbamate, O-thiocarbamate, N-thiocarbamate, C-amido, N-amido or amino, as these terms are defined hereinabove or hereinbelow.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, alkyl, cycloalkyl, aryl, heteroaryl, halo, trihalomethyl, hydroxy, alkoxy, aryloxy, thiohydroxy, thioalkoxy, thioaryloxy, cyano, nitro, carbonyl, thiocarbonyl, C-carboxy, O-carboxy, O-carbamate, N-carbamate, O-thiocarbamate, N-thiocarbamate, sulfinyl, sulfonyl, C-amido, N-amido or amino, as these terms are defined hereinabove or hereinbelow.

The term “halo” describes a fluorine, chlorine, bromine or iodine atom.

The term “amino”, as is defined hereinabove with respect to an “amine” or an “amino group”, is used herein to describe an —NR′R″, wherein R′ and R″ are each independently hydrogen, alkyl, cycloalkyl, aryl, heteroaryl or heterocyclic, as these terms are defined hereinabove.

Hence, the terms “alkylamino”, “arylamino”, “cycloalkylamino”, “heteroalicyclic amino” and “heteroarylamino” describe an amino group, as defined hereinabove, wherein at least one of R′ and R″ thereof is alkyl, aryl, cycloalkyl, heterocyclic and heteroaryl, respectively.

The term “hydroxy” describes an —OH group.

An “alkoxy” describes both an —O-alkyl and an —O-cycloalkyl group, as defined herein.

An “aryloxy” describes both an —O-aryl and an —O-heteroaryl group, as defined herein.

The term “azo” describes a —N═N group.

A “C-amido” describes a —C(═O)—NR′R″ group, where R′ and R″ are as defined hereinabove.

An “N-amido” describes a R′C(═O)—NR″— group, where R′ and R″ are as defined hereinabove.

An “ammonium” describes an —N⁺HR′R″ group, where R′ and R″ are as defined hereinabove.

The term “thiohydroxy” describes a —SH group.

The term “thioalkoxy” describes both a —S-alkyl group and a —S-cycloalkyl group, as defined hereinabove.

The term “thioaryloxy” describes both a —S-aryl and a —S-heteroaryl group, as defined hereinabove.

A “sulfinyl” describes a —S(═O)—R group, where R can be, without limitation, alkyl, cycloalkyl, aryl and heteroaryl as these terms are defined hereinabove.

A “sulfonyl” describes a —S(═O)₂—R group, where R is as defined hereinabove.

A “S-sulfonamido” is a —S(═O)₂—NR′R″ group, with R′ and R″ as defined hereinabove.

A “N-sulfonamido” is an R′(S′O)₂—NR″— group, with R′ and R″ as defined hereinabove.

A “phosphonyl” is a —O—P(═O)(OR′)—R″ group, with R′ and R″ as defined hereinabove.

A “phosphinyl” is a —PR′R″ group, with R′ and R″ as defined hereinabove.

A “phosphonium” is a —P⁺R′R″R′″, where R′ and R″ are as defined hereinabove and R′″ is defined as either R′ or R″.

The term “carbonyl” describes a —C(═O)—R group, where R is hydrogen, alkyl, cycloalkyl, aryl, heteroaryl (bonded through a ring carbon) or heteroalicyclic (bonded through a ring carbon) as defined hereinabove.

A “thiocarbonyl” describes a —C(═S)—R group, where R is as defined hereinabove with respect to the term “carbonyl”.

A “C-carboxy” describes a —C(═O)—O—R groups, where R is as defined hereinabove with respect to the term “carbonyl”.

An “O-carboxy” group refers to a RC(═O)—O— group, where R is as defined hereinabove with respect to the term “carbonyl”.

A “carboxylic acid” is a C-carboxy group in which R is hydrogen.

A “C-thiocarboxy” is a —C(═S)—O—R groups, where R is as defined hereinabove with respect to the term “carbonyl”.

An “O-thiocarboxy” group refers to an R—C(═S)—O— group, where R is as defined hereinabove with respect to the term “carbonyl”.

The term “O-carbamate” describes an —OC(═O)—NR′R″ group, with R′ and R″ as defined hereinabove.

A “N-carbamate” describes a R′—O—C(═O)—NR″— group, with R′ and R″ as defined hereinabove.

An “O-thiocarbamate” describes an —O—C(═S)—NR′R″ group, with R′ and R″ as defined hereinabove.

A “N-thiocarbamate” describes a R′OC(═S)NR″— group, with R′ and R″ as defined hereinabove.

The term “urea” describes a —NR′—C(═O)—NR′R″ group, with R′, R″ and R′″ as defined hereinabove.

The term “thiourea” describes a —NR′—C(═S)—NR′R″ group, with R′, R″ and R′″ as defined hereinabove.

The term “borate” describes an —O—B—(OR)₂ group, with R as defined hereinabove.

The term “borane” describes a —B—R′R″ group, with R′ and R″ as defined hereinabove.

The term “boraza” describes a —B(R′)(NR″R′″) group, with R′, R″ and R′″ as defined hereinabove.

The tern “silyl” describes a —SiR′R″R′″, with R′, R″ and R′″ as defined herein.

The term “siloxy” is a —Si—(OR)₃, with R as defined hereinabove.

The term “silaza” describes a —Si—(NR′R″)₃, with R′ and R″ as defined herein.

The term “aquo” describes a H₂O group.

The term “alcohol” describes a ROH group, with R as defined hereinabove.

The term “peroxo” describes an —OOR group, with R as defined hereinabove.

As used herein, an “amine oxide” is a —N(═O)R′R″R′″ group, with R′, R″ and R′″ as defined herein.

A “hydrazine” is a —NR′—NR″R′″ group, with R′, R″ and R′″ as defined herein.

Hence, “alkyl hydrazine” and “aryl hydrazine” describe a hydrazine where R′ is an alkyl or an aryl, respectively, and R″ and R′″ are as defined hereinabove.

The term “nitric oxide” is a —N═O group.

The term “cyano” is a —C≡N group.

A “cyanate” is an —O—C≡N group.

A “thiocyanate” is a “—S—C≡N group.

An “isocyanate” is a —N═C═O group.

An “isothiocyanate” is a —N═C═S group.

The terms “alkyl nitrile” and “aryl nitrile” describe a —R—C≡N group, where R is an alkyl or an aryl, respectively.

The terms “alkyl isonitrile” and “aryl isonitrile” describe a R—N≡C— group, where R is an alkyl or aryl, respectively.

A “nitrate” or “nitro” is a —NO₂ group.

A “nitrite” is an —O—N═O group.

An “azido” is a N₃⁺ group.

An “alkyl sulfonic acid” and an “aryl sulfonic acid” describe a —R—SO₂—OH group, with R being an alkyl or an aryl, respectively.

An “alkyl sulfoxide”, an “aryl sulfoxide” and an “alkyl aryl sulfoxide” describe a —R′S(═O)R″ group, where R′ and R″ are each an alkyl, R′ and R″ are each an aryl and where R′ is and alkyl and R″ is an aryl, respectively.

An “alkyl sulfenic acid” and “aryl sulfenic acid” describe a —R—S—OH group, where R is an alkyl or an aryl, respectively.

An “alkyl sulfinic acid” and “aryl sulfinic acid” describe a —R—S(═O)—OH group where R is an alkyl or an aryl, respectively.

As used herein, the terms “alkyl carboxylic acid” and “aryl carboxylic acid” describe a —R—C(═O)—OH group, where R is an alkyl or an aryl, respectively.

An “alkyl thiol carboxylic acid” and an —aryl thiol carboxylic acid” describe a —R—C(═O)—SH group, where R is an alkyl or an aryl, respectively.

An “alkyl thiol thiocarboxylic acid” and an “aryl thiol thiocarboxylic acid” describe a —R—C(═S)—SH group, where R is an alkyl or an aryl, respectively.

A “sulfate” is a —O—SO₂—OR′ group, with R′ as defined hereinabove.

A “sulfite” group is a —O—S(═O)—OR′ group, with R′ as defined hereinabove.

A “bisulfite” is a sulfite group, where R′ is hydrogen.

A “thiosulfate” is an —O—SO₂—SR′ group, with R′ as defined hereinabove.

A “thiosulfite” group is an —O—S(═O)—SR′ group, with R′ as defined hereinabove.

The terms “alkyl/aryl phosphine” describe a —R—PH₂ group, with R being an alkyl or an aryl, respectively, as defined above.

The terms “alkyl and/or aryl phosphine oxide” describe a —R′—PR″₂(═O) group, with R′ and R″ being an alkyl and/or an aryl, as defined hereinabove.

The terms “allyl and/or aryl phosphine sulfide” describe a —R′—PR″₂(═S) group, with R′ and R″ being an alkyl and/or an aryl, as defined hereinabove.

The terms “alkyl/aryl phosphonic acid” describe a —R′—P(═O)(OH)₂ group, with R′ being an alkyl or an aryl as defined above.

The terms “alkyl/aryl phosphinic acid” describes a —R′—P(OH)₂ group, with R′ being an alkyl or an aryl as defined above.

A “phosphate” is a —O—P(═O)(OR′)(OR″) group, with R′ and R″ as defined hereinabove.

A “hydrogen phosphate” is a phosphate group, where R′ is hydrogen.

A “dihydrogen phosphate” is a phosphate group, where R′ and R″ are both hydrogen.

A “thiophosphate” is a —S—P(═O)(OR′)₂ group, with R′ as defined hereinabove.

A “phosphite” is an —O—P(OR′)₂ group, with R′ as defined hereinabove.

A “pyrophosphite” is an —O—P—(OR′)—O—P(OR″)₂ group, with R′ and R″ as defined hereinabove.

A “triphosphate” describes an —OP(═O)(OR′)—O—P(═O)(OR″)—O—P(═O)(OR′″)₂, with R′, R″ and R′″ are as defined hereinabove.

As used herein, the term “guanidine” describes a —R′NC(═N)—NR″R′″ group, with R′, R″ and R′″ as defined herein.

The term “S-dithiocarbamate” describes a —SC(═S)—NR′R″ group, with R′ and R″ as defined hereinabove.

The term “N-dithiocarbamate” describes an R′SC(═S)NR″— group, with R′ and R″ as defined hereinabove.

A “bicarbonate” is an —O—C(═O)—O⁻ group.

A “carbonate” is an —O—C(═O)—OH group.

A “perchlorate” is an —O—Cl(═O)₃ group.

A “chlorate” is an —O—Cl(═O) group.

A “chlorite” is an —O—Cl(═O) group.

A “hypochlorite” is an —OCl group.

A “perbromate” is an —O—Br(═O)₃ group.

A “bromate” is an —O—Br(═O)₂ group.

A “bromite” is an —O—Br(═O) group.

A “hypobromite” is an —OBr group.

A “periodate” is an —O—I(═O)₃ group.

A “iodate” is an —O—I(═O)₂ group.

The term “tetrahalomanganate” describes MnCl₄, MnBr₄ and MnI₄.

The term “tetrafluoroborate” describes a —BF₄ group.

A “tetrafluoroantimonate” is a SbF₆ group.

A “hypophosphite” is a —P(OH)₂ group.

The term “metaborate” describes the group 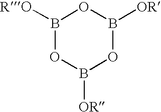
where R′, R″ and R′″ are as defined hereinabove.

The terms “tetraalkyl/tetraaryl borate” describe a R′B⁻ group, with R′ being an alkyl or an aryl, respectively, as defined above.

A “tartarate” is an —OC(═O)—CH(OH)—CH(OH)—C(═O)OH group.

A “salycilate” is the group

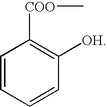

A “succinate” is an —O—C(═O)—(CH₂)₂—COOH group.

A “citrate” is an —O—C(═O)—CH₂—CH(OH)(COOH)—CH₂—COOH group.

An “ascorbate” is the group

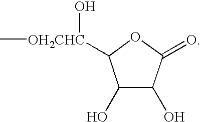

A “saccharirate” is an oxidized saccharide having two carboxylic acid group.

The term “amino acid” as used herein includes natural and modified amino acids and hence includes the 21 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids which are linked via a peptide bond or a peptide bond analog to at least one addition amino acid as this term is defined herein.

A “hydroxamic acid” is a —C(═O)—NH—OH group.

A “thiotosylate” is the group

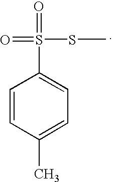

Similarly, each of the alkylene chains B₁ . . . Bn independently has a general formula III: 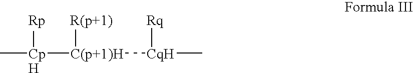
wherein p is an integer that equals 0 or g+1 and q is an integer from g+2 to g+20.

Hence, each of the alkylene chains B₁ . . . Bn is comprised of a plurality of carbon atoms Cp, Cp+1, Cp+2 . . . , Cq−1 and Cq, substituted by the respective Rp, Rp+1, Rp+2 . . . , Rq−1 and Rq groups. Preferably, each of the alkylene chains B₁ . . . Bn includes 2-20 carbon atoms, more preferably 2-10, and most preferably 2-6 carbon atoms.

As is defined hereinabove, in cases where p equals 0, the component —CpH(Rp)- is absent from the structure. In cases where p equals g+1, it can be either 1 or 4-11. The integer q can be either 2 or 5-20.

Each of the substituents Rp, Rp+1 . . . Rn can be any of the substituents described hereinabove with respect to R₁, R₂ and Rg.

Hence, a preferred linear polyamine according to the present invention includes two or more alkylene chains. The alkylene chains are interrupted therebetween by a heteroatom and each is connected to a heteroatom at one end thereof. Preferably, each of the alkylene chains include at least two carbon atoms, so as to enable the formation of a stable chelate between the heteroatoms and the copper ion.

The linear polyamine delineated in Formula I preferably includes at least one chiral carbon atom. Hence, at least one of C₁, C₂ and Cg in the alkylene chain A and/or at least one of Cp, Cp+1 and Cq in the alkylene chain B is chiral.

A preferred linear polyamine according to the present invention is tetraethylenepentamine. Other representative examples of preferred linear polyamines usable in the context of the present invention include, without limitation, ethylendiamine, diethylenetriamine, triethylenetetramine, triethylenediamine, aminoethylethanolamine, pentaethylenehexamine, triethylenetetramine, N,N′-bis(3-aminopropyl)-1,3-propanediamine, and N,N′-Bis(2-animoethyl)-1,3 propanediamine.

In cases where the polyamine chelator is a cyclic polyamine, the polyamine can have a general formula IV: 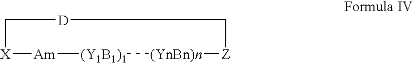
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; B₁ and Bn are each independently an alkylene chain having between 1 and 20 substituted and/or non-substituted carbon atoms; and D is a bridging group having a general formula V:
U—W—V Formula V
whereas U and V are each independently selected from the group consisting of substituted hydrocarbon chain and non-substituted hydrocarbon chain; and W is selected from the group consisting of amide, ether, ester, disulfide, thioether, thioester, imine and alkene, provided that at least one of said X, Z, Yi and Yn is a —NH group and/or at least one of said carbon atoms in said alkylene chains is substituted by an amine group.

Optionally, the cyclic polyamine has one of the general formulas VI-X: 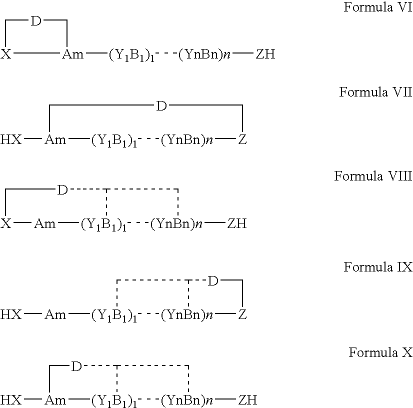
wherein m, n, X, Y₁, Yn, Z, A, B and D are as described above and further wherein should the bridging group D is attached at one end to A (Formulas VI, VII and X), U or V are being attached to one carbon atom in the alkylene chain and should D is attached at one end to B1 or Bn (Formulas VIII, IX and X), U or V are being attached to one carbon atom in the alkylene chain.

Hence, a preferred cyclic polyamine according to the present invention includes two or more alkylene chains, A, B₁ . . . Bn, as is detailed hereinabove with respect to the linear polyamine. The alkylene chains can form a cyclic structure by being connected, via the bridging group D, between the ends thereof, namely between the heteroatoms X and Z (Formula IV). Optionally, the alkylene chains can form a conformationally restricted cyclic structure by being connected, via the bridging group D, therebetween (formula X). Further optionally, a conformationally restricted cyclic structure can be formed by connecting one alkylene chain to one terminal heteroatom (X or Z, Formulas VI-IX).

As is described hereinabove, in cases where the cyclic structure is formed by connecting one alkylene chain to one terminal heteroatom, as is depicted in Formulas VI-IX, the bridging group D connects a terminal heteroatom, namely X or Z, and one carbon atom in the alkylene chains A and B₁ . . . Bn. This carbon atom can be anyone of C₁, C₂, Cg, Cp, Cp+1 and Cq described hereinabove.

As is further described hereinabove, the cyclic structure is formed by the bridging group D, which connects two components in the structure. The bridging group D has a general formula U—W—V, where each of U and V is a substituted or non-substituted hydrocarbon chain.

As used herein, the phrase “hydrocarbon chain” describes a plurality of carbon atoms which are covalently attached one to another and are substituted, inter alia, by hydrogen atoms. The hydrocarbon chain can be saturated, unsaturated, branched or unbranched and can therefore include one or more alkyl, alkenyl, alkynyl, cycloalkyl and aryl groups and combinations thereof.

The length of the hydrocarbon chains, namely the number of carbon atoms in the chains, is preferably determined by the structure of the cyclic polyamine, such that on one hand, the ring tension of the formed cyclic structure would be minimized and on the other hand, an efficient chelation with the copper ion would be achieved.

When the hydrocarbon chain is substituted, the substituents can be any one or combinations of the substituents described hereinabove with respect to R₁, R₂ and Rg in the linear polyamine.

The two hydrocarbon chains are connected therebetween by the group W, which can be amide, ether, ester, disulfide, thioether, thioester, imine and alkene.

As used herein, the term “ether” is an —O— group.

The term “ester” is a —C(═O)—O— group.

A “disulfide” is a —S—S— group.

A “thioether” is a —S— group.

A “thioester” is a —C(═O)—S— group.

An “imine” is a —C(═NH)— group.

An “alkene” is a —CH═CH— group.

The bridging group D is typically formed by connecting reactive derivatives of the hydrocarbon chains U and V, so as to produce a bond therebetween (W), via well-known techniques, as is described, for example, in U.S. Pat. No. 5,811,392.

As is described above with respect to the linear polyamine, the cyclic polyamine must include at least one amine group, preferably at least two amine groups and more preferably at least four amine groups, so as to form a stable copper chelate.

A preferred cyclic polyamine according to the present invention is cyclam (1,4,8,11-tetraazacyclotetradecane).

As is described hereinabove, the polyamine chelator of the present invention can further include a multimeric combination of one or more linear polyamine(s) and one or more cyclic polyamine(s). Such a polyamine chelator can therefore be comprised of any combinations of the linear and cyclic polyamines described hereinabove.

Preferably, such a polyamine chelator has a general Formula XI:
{(E₁)_{f}-[Q₁-(G₁)_{g}]}ₕ-{(E₂)ᵢ-[Q₂-(G₂)ⱼ]}ₖ- . . . -{(Eₙ)₁-[Qₙ-(Gₙ)}ₜ Formula XI
wherein n is an integer greater than 1; each of f, g, h, i, j, k, l, o and t is independently an integer from 0 to 10; each of E₁, E₂ and En is independently a linear polyamine, as is described hereinabove; each of G₁, G₂ and Gn is independently a cyclic polyamine as is described hereinabove; and each of Q₁, Q₂ and Qn is independently a linker linking between two of said polyamines, provided that at least one of said Q₁, Q₂ and Qn is an amine group and/or at least one of said linear polyamine and said cyclic polyamine has at least one free amine group.

Each of E₁, E₂ and En in Formula XI represent a linear polyamine as is described in detail hereinabove, while each of G₁, G₂ and Gn represents a cyclic polyamine as is described in detail hereinabove.

The polyamine described in Formula XI can include one or more linear polyamine(s), each connected to another linear polyamine or to a cyclic polyamine.

Each of the linear or cyclic polyamines in Formula XI is connected to another polyamine via one or more linker(s), represented by Q₁, Q₂ and Qn in Formula XI.

Each of the linker(s) Q₁, Q₂ and Qn can be, for example, alkylene, alkenylene, alkynylene, arylene, cycloalkylene, hetroarylene, amine, azo, amide, sulfonyl, sulfinyl, sulfonamide, phosphonyl, phosphinyl, phosphonium, ketoester, carbonyl, thiocarbonyl, ester, ether, thioether, carbamate, thiocarbamate, urea, thiourea, borate, borane, boroaza, silyl, siloxy and silaza.

As used herein, the term “alkenylene” describes an alkyl group which consists of at least two carbon atoms and at least one carbon-carbon double bond.

The term “alkynylene” describes an alkyl group which consists of at least two carbon atoms and at least one carbon-carbon triple bond.

The term “cycloalkylene” describes an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane.

The term “arylene” describes an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted.

The term “heteroarylene” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted.

As used in the context of the linker of the present invention, the term “amine” describes an —NR′—, wherein R′ can be hydrogen, alkyl, cycloalkyl, aryl, heteroaryl or heterocyclic, as these terms are defined hereinabove.

As is further used in the context of the linker of the present invention, the term “azo” describes a —N═N— group.

The term “amide” describes a —C(═O)—NR′— group, where R′ is as defined hereinabove.

The term “ammonium” describes an —N⁺HR′— group, where R′ is as defined hereinabove.

The term “sulfinyl” describes a —S(═O)— group.

The term “sulfonyl” describes a —S(═O)₂— group.

The term “sulfonamido” describes a —S(═O)₂—NR′— group, with R′ as defined hereinabove.

The term “phosphonyl” describes a —O—P(═O)(OR′)— group, with R′ as defined hereinabove.

The term “phosphinyl” describes a —PR′— group, with R′ as defined hereinabove.

The term “phosphonium” is a —P⁺R′R″, where R′ and R″ are as defined hereinabove.

The term “ketoester” describes a —C(═O)—C(═O)—O— group.

The term “carbonyl” describes a —C(═O)— group.

The term “thiocarbonyl” describes a —C(═S)— group.

The term “carbamate” describes an —OC(═O)—NR′— group, with R′ as defined hereinabove.

The term “thiocarbamate” describes an —OC(═S)—NR— group, with R′ as defined hereinabove.

The term “urea” describes an —NR′—C(═O)—NR″— group, with R′ and R″ and as defined hereinabove.

The term “thiourea” describes a —NR′—C(═S)—NR′— group, with R′ and R″ as defined hereinabove.

The term “borate” describes an —O—B—(OR)— group, with R as defined hereinabove.

The term “borane” describes a —B—R—′— group, with R as defined hereinabove.

The term “boraza” describes a —B(NR′R″)— group, with R′ and R″ as defined hereinabove.

The term “silyl” describes a —SiR′R″—, with R′ and R″ as defined herein.

The term “siloxy” is a —Si—(OR)₂—, with R as defined hereinabove.

The term “silaza” describes a —Si—(NR′R″)₂—, with R′ and R″ as defined herein.

It should be noted that all the terms described hereinabove in the context of the linker of the present invention are the same as described above with respect to the substituents. However, in distinction from the substituent groups, which are connected to a component at one end thereof, the linker groups are connected to two components at two sites thereof and hence, these terms have been redefined with respect to the linker.

As has been mentioned hereinabove, according to the presently most preferred embodiment of the present invention, the polyamine chelator is tetraethylenepentamine (TEPA). However, other preferred polyamine chelators include, without limitation, ethylendiamine, diethylenetriamine, triethylenetetramine, triethylenediamine, aminoethylethanolamine, aminoethylpiperazine, pentaethylenehexamine, triethylenetetramine, captopril, penicilamine, N,N′-bis(3-aminopropyl)-1,3-propanediamine, N,N′-Bis(2-animoethyl)-1,3-propanediamine, 1,7-dioxa-4,10-diazacyclododecane, 1,4,8,11-tetraazacyclotetradecane-5,7-dione, 1,4,7-triazacyclononane, 1-oxa-4,7,10-triazacyclododecane, 1,4,8,12-tetraazacyclopentadecane and 1,4,7,10-tetraazacyclododecane.

The above listed preferred chelators are known in their high affinity towards copper ions. However, these chelators are further beneficially characterized by their substantial affinity also towards other transition metals, as is described by Ross and Frant [22], which is incorporated by reference as if fully set forth herein.

All the polyamine chelators described hereinabove can be either commercially obtained or can be synthesized using known procedures such as described, for example, in: T. W. Greene (ed.), 1999 (“Protective Groups in Organic Synthesis” 3ed Edition, John Wiley & Sons, Inc., New York 779 pp); or in: R. C. Larock and V. C. H. Wioley, “Comprehensive Organic Transformations—A Guide to Functional Group Preparations”, (1999) 2nd Edition.

The copper chelate can be provided to the cell culture medium. The final concentrations of copper chelate may be, depending on the specific application, in the micromolar or millimolar ranges, for example, within about 0.1 μM to about 100 mM, preferably within about 4 μM to about 50 mM, more preferably within about 5 μM to about 40 mM. As is described hereinabove, the copper chelate is provided to the cells so as to maintain the free copper concentration of the cells substantially unchanged during cell expansion.

The stem and/or progenitor cells used in the present invention can be of various origin. According to a preferred embodiment of the present invention, the stern and/or progenitor cells are derived from a source selected from the group consisting of hematopoietic cells, umbilical cord blood cells, G-CSF mobilized peripheral blood cells, bone marrow cells, hepatic cells, pancreatic cells, neural cells, oligodendrocyte cells, skin cells, embryonal stem cells, muscle cells, bone cells, mesenchymal cells, chondrocytes and stroma cells. Methods of preparation of stem cells from a variety of sources are well known in the art, commonly selecting cells expressing one or more stem cell markers such as CD34, CD133, etc, or lacking markers of differentiated cells. Selection is usually by FACS, or immunomagnetic separation, but can also be by nucleic acid methods such as PCR (see Materials and Experimental Methods hereinbelow). Embryonic stem cells and methods of their retrieval are well known in the art and are described, for example, in Trounson A O (Reprod Fertil Dev (2001) 13: 523), Roach M L (Methods Mol Biol (2002) 185: 1), and Smith A G (Annu Rev Cell Dev Biol (2001) 17:435). Adult stem cells are stem cells, which are derived from tissues of adults and are also well known in the art. Methods of isolating or enriching for adult stem cells are described in, for example, Miraglia, S. et al. (1997) Blood 90: 5013, Uchida, N. et al. (2000) Proc. Natl. Acad. Sci. USA 97: 14720, Simmons, P. J. et al. (1991) Blood 78: 55, Prockop D J (Cytotherapy (2001) 3: 393), Bohmer R M (Fetal Diagn Ther (2002) 17: 83) and Rowley S D et al. (Bone Marrow Transplant (1998) 21: 1253), Stem Cell Biology Daniel R. Marshak (Editor) Richard L. Gardner (Editor), Publisher: Cold Spring Harbor Laboratory Press, (2001) and Hematopoietic Stem Cell Transplantation. Anthony D. Ho (Editor) Richard Champlin (Editor), Publisher: Marcel Dekker (2000).

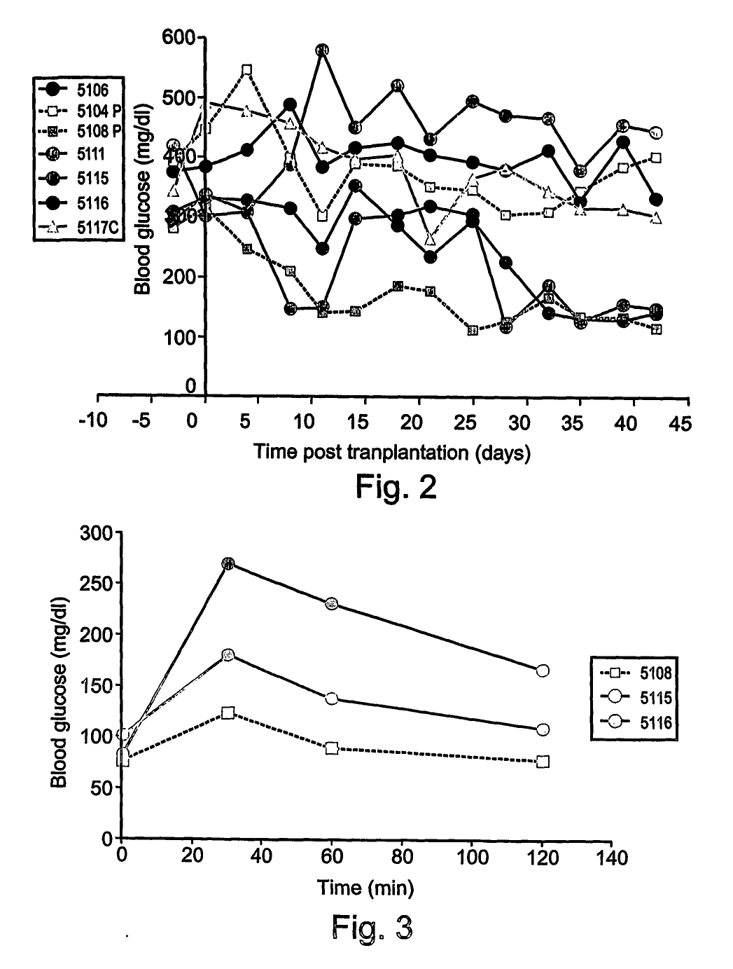

Ianus et al. (J Clin. Invest 2003;1l 1:843-850) demonstrated that nucleated bone marrow cells from GFP-transgenic mice, when implanted into wild type mice, produced pancreatic islet cells expressing GFP. However, the bone marrow cell fraction implanted was not expanded ex-vivo prior to implantation. As detailed in the Examples section hereinbelow, while reducing the present invention to practice, it was surprisingly uncovered that unselected cells from the total nucleated cell (TNC) fraction of cord blood can be expanded ex-vivo while inhibiting differentiation thereof, and used for restoring pancreatic function in STZ-diabetic mice by direct transplantation (see Example II, and FIGS. 2 and 3). Thus, in one embodiment of the present invention, the population of cells comprising stem and/or progenitor cells is unselected mononuclear cells. PCT IL03/00681 to Peled, et al, which is incorporated by reference as if fully set for herein, discloses the use of molecules such as copper chelators, copper chelates and retinoic acid receptor (RAR) antagonists which are capable of repressing differentiation and stimulating and prolonging proliferation of hematopoietic stem cells when the source of cells includes the entire fraction of mononuclear blood cells, namely non-enriched stem cells.

As used herein, the phrase “hematopoietic mononuclear cells” refers to the entire repertoire of white blood cells present in a blood sample, usually hematopoietic mononuclear cells which comprise a major fraction of hematopoietic committed cells and a minor fraction of hematopoietic stem and progenitor cells. In a healthy human being, the white blood cells comprise a mixture of hematopoietic lineages committed and differentiated cells (typically over 99% of the mononuclear cells are lineages committed cells) including, for example: Lineage committed progenitor cells CD34⁺ CD33⁺ (myeloid committed cells), CD34⁺ CD3⁺ (lymphoid committed cells) CD34⁺ CD41⁺ (megakaryocytic committed cells) and differentiated cells—CD34⁻CD33⁺ (myeloids, such as granulocytes and monocytes), CD34⁻CD3⁺, CD34⁻CD19⁺ (T and B cells, respectively), CD34⁻CD41⁺ (megakaryocytes), and hematopoietic stem and early progenitor cells such as CD34⁺ Lineage negative (Lin⁻), CD34-Lineage negative CD34⁺CD38⁻ (typically less than 1%).

The phrase “hematopoietic mononuclear cells which comprise a major fraction of hematopoietic committed cells and a minor fraction of hematopoietic stem and progenitor cells” is used herein to describe any portion of the white blood cells fraction, in which the majority of the cells are hematopoietic committed cells, while the minority of the cells are hematopoietic stem and progenitor cells, as these terms are further defined hereinunder.

Hematopoietic mononuclear cells are typically obtained from a blood sample by applying the blood sample onto a Ficoll-Hypaque layer and collecting, following density-cushion centrifugation, the interface layer present between the Ficoll-Hypaque and the blood serum, which interface layer essentially entirely consists of the white blood cells present in the blood sample.

Presently, hematopoietic stem cells are obtained by further enrichment of the hematopoietic mononuclear cells obtained by differential density centrifugation as described above. This further enrichment process is typically performed by immuno-separation such as immunomagnetic-separation or FACS and results in a cell fraction that is enriched for hematopoietic stern cells (for detailed description of enrichment of hematopoietic stem cells, see Materials and Experimental Procedures in the Examples section hereinbelow).

Hence, using hematopoietic mononuclear cells as a direct source for obtaining expanded population of hematopoietic stem cells circumvents the need for stem cell enrichment prior to expansion, thereby substantially simplifying the process in terms of both efficiency and cost.

According to one aspect of the present invention, the ex-vivo expansion of populations of stem cells, according to the features described hereinabove, can be utilized for expanding a population of renewable stem cells ex-vivo for implanting the cells in an endodermally-derived organ of a subject in need thereof.

As used herein, the term “endodermally-derived” is defined as originating from the embryonic endoderm. Endodermally derived organs are the epithelial cells of the alimentary canal, liver, pancreas, lung, and thyroid gland.

Implanting can be by means of direct injection into the organ, injection into the bloodstream, intraperitoneal injection, etc. Suitable methods of implantation can be determined by monitoring the homing of the implanted cells to the desired organ, the expression of desired organ-specific genes or markers, and the function of the endodermally-derived organ of the subject. In the pancreas, for example, maintenance of euglycemia, secretion of insulin and/or C peptide can be a measure of the restoration of function to a diabetic host animal following cell replacement therapy as disclosed hereinbelow. In the liver, for example, albumin synthesis can be monitored.

It will be appreciated that ex-vivo expanded stem and/or progenitor cells can be implanted into endodermally derived organs in order to provide the cells with an environment conducive to differentiation into cells expressing characters specific to cells of the endodermal organs, and that cells thus differentiated can be removed, reisolated, and further reimplanted into other organs, or other subjects.

Recent studies with transplantation of stem cells for repopulation of injured or diseased liver or pancreas have indicated that enrichment of the stem and/or progenitor cells for cells having endodermal stem cell characteristics, prior to their implantation, results in increased success of repopulation and restoration of organ function. For example, Hess et al. (Nature Biotech Advance Online Publication 22 Jun. 2003) describes the selection of bone marrow stem cells expressing the pancreatic islet marker c-kit, and repopulation of the pancreas in STZ-diabetic host mice. McKay et al selected nestin-positive neuroendocrine precursor cells, and exposed the cells to combinations of soluble factors to promote the expression of islet cell phenotype. In similar experiments, Kakinuma et al (Stem Cells 2003;21:217-227) implanted an unexpanded fraction of murine cord blood cells, exposed to hepatic factors FGF-1, FGF-2, LIF, SCF, HGF and OSM, into SCID hepatectomized mice, effectively repopulating the liver parenchyma in the hepatectomized hosts. The repopulating bone marrow derived cells were also shown to produce albumin. Thus, in one embodiment of the present invention, following the step of culturing the stem and/or progenitor cells under conditions allowing for cell proliferation and, at the same time, substantially inhibiting differentiation thereof, enrichment of stem cells having an endodermal phenotype is performed.

As used herein, the term “endodermal cell phenotype” is defined as the detectable presence of any identifying characteristic of endodermal cells, including morphological, genetic markers, metabolic, etc. characteristics. Such enrichment can be effected by providing at least one hepatic growth factor, and/or sodium butyrate. Suitable hepatic growth factors include, but are not limited to FGF-1, FGF-2, LIF, OSM, HGM, FBS, HGF, EGF and SCF. Selection, by morphology, FACS or immunomagnetic selection, for oval cells or cells expressing endodermal markers such as OC3, c-kit, PDX-1, PDX-4, etc. can also be used. Suitable hepatocyte cell markers, for example, include HNF-3β, GATA4, CK-19, transerythrin, albumin and urea synthesis, cytochrome p450 synthesis, and the presence of binucleated cells. Suitable pancreatic cell markers include formation of three-dimensional islet cell-like clusters and expression of pancreatic islet cell differentiation-related transcripts such as PDX-1, PAX-4, PAX-6, Nkx2.2 and Nkx6.1, insulin I, insulin II, glucose transporter 2, and glucagon, and islet-specific hormones detectable by immunocytochemistry such as insulin, glucagon, and pancreatic polypeptide. Wang et al (PNAS USA, 2003;100:11881-88) discloses the repopulation of injured mouse liver with preselected hepatic “oval” cells. Yang et al (PNAS USA 2002;99:8078-83) exposed cultured adult hepatic “oval” cells to high concentrations of glucose, leading to the expression of pancreas islet cell phenotype before implantation into diabetic host mice. Ber et al (JBC 2003;278:31950-57) forced PDX-1 expression in liver cells in-vivo to produce transdifferentiation of hepatocytes or hepatocyte progenitors to cells expressing a pancreatic phenotype. Additional methods for enrichment and selection of cells having an endodermal cell phenotype are disclosed in US Patent Application No. 20020182728 to Ramiya et al.

As described hereinabove, and detailed in the Examples section hereinbelow, prior to implantation the stem and/or progenitor cells are cultured ex-vivo under conditions allowing for cell proliferation and, at the same time, substantially inhibiting differentiation thereof. According to preferred embodiments of the present invention, providing the stem cells with the conditions for ex-vivo cell proliferation comprises providing the cells with nutrients and with cytokines. Preferably, the cytokines are early acting cytokines, such as, but not limited to, stem cell factor, FLT3 ligand, interleukin-1, interleukin-2, interleukin-3, interleukin-6, interleukin-10, interleukin-12, tumor necrosis factor-α and thrombopoietin. It will be appreciated in this respect that novel cytokines are continuously discovered, some of which may find uses in the methods of cell expansion of the present invention.

Late acting cytokines can also be used. These include, for example, granulocyte colony stimulating factor, granulocyte/macrophage colony stimulating factor, erythropoietin, FGF, EGF, NGF, VEGF, LIF, Hepatocyte growth factor and macrophage colony stimulating factor.

The present invention can be used for gene therapy. Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (i) ex-vivo or cellular gene therapy; and (ii) in vivo gene therapy. In ex-vivo gene therapy cells are removed from a patient, and while being cultured are treated in-vitro. Generally, a functional replacement gene is introduced into the cells via an appropriate gene delivery vehicle/method (transfection, transduction, homologous recombination, etc.) and an expression system as needed and then the modified cells are expanded in culture and returned to the host/patient. These genetically re-implanted cells have been shown to express the transfected genetic material in situ.

Hence, in one embodiment of the present invention, the stem and/or progenitor cells are genetically modified cells. In a preferred embodiment, genetically modifying the cells is effected by a vector, which comprises the exogene or transgene, which vector is, for example, a viral vector or a nucleic acid vector. Many viral vectors suitable for use in cellular gene therapy are known, examples are provided hereinbelow. Similarly, a range of nucleic acid vectors can be used to genetically transform the expanded cells of the invention, as is further described below.

Accordingly, the expanded cells of the present invention can be modified to express a gene product. As used herein, the phrase “gene product” refers to proteins, peptides and functional RNA molecules. Generally, the gene product encoded by the nucleic acid molecule is the desired gene product to be supplied to a subject. Examples of such gene products include proteins, peptides, glycoproteins and lipoproteins normally produced by an organ of the recipient subject. For example, gene products which may be supplied by way of gene replacement to defective organs in the pancreas include insulin, amylase, protease, lipase, trypsinogen, chymotrypsinogen, carboxypeptidase, ribonuclease, deoxyribonuclease, triaclyglycerol lipase, phospholipase A₂, elastase, and amylase; gene products normally produced by the liver include blood clotting factors such as blood clotting Factor VIII and Factor DC, UDP glucuronyl transferae, ornithine transcarbanoylase, and cytochrome p450 enzymes, and adenosine deaminase, for the processing of serum adenosine or the endocytosis of low density lipoproteins; gene products produced by the thymus include serum thymic factor, thymic humoral factor, thymopoietin, and thymosin α₁; gene products produced by the digestive tract cells include gastrin, secretin, cholecystokinin, somatostatin, serotinin, and substance P.

Alternatively, the encoded gene product is one, which induces the expression of the desired gene product by the cell (e.g., the introduced genetic material encodes a transcription factor, which induces the transcription of the gene product to be supplied to the subject).

In still another embodiment, the recombinant gene can provide a heterologous protein, e.g., not native to the cell in which it is expressed. For instance, various human MHC components can be provided to non-human cells to support engraftment in a human recipient. Alternatively, the transgene is one, which inhibits the expression or action of a donor MHC gene product.

A nucleic acid molecule introduced into a cell is in a form suitable for expression in the cell of the gene product encoded by the nucleic acid. Accordingly, the nucleic acid molecule includes coding and regulatory sequences required for transcription of a gene (or portion thereof) and, when the gene product is a protein or peptide, translation of the gene acid molecule include promoters, enhancers and polyadenylation signals, as well as sequences necessary for transport of an encoded protein or peptide, for example N-terminal signal sequences for transport of proteins or peptides to the surface of the cell or secretion.

Nucleotide sequences which regulate expression of a gene product (e.g., promoter and enhancer sequences) are selected based upon the type of cell in which the gene product is to be expressed and the desired level of expression of the gene product. For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements, which are known in the art, include upstream regions from the dystrophin gene (Klamut et al., (1989) Mol. Cell Biol. 9: 2396), the creatine kinase gene (Buskin and Hauschka, (1989) Mol. Cell Biol. 9: 2627) and the troponin gene (Mar and Ordahl, (1988) Proc. Nat. Acad. Sci. USA. 85: 6404). Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters).

Alternatively, a regulatory element, which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs.

Alternatively, a regulatory element, which provides inducible expression of a gene linked thereto, can be used. The use of an inducible regulatory element (e.g., an inducible promoter) allows for modulation of the production of the gene product in the cell. Examples of potentially useful inducible regulatory systems for use in eukaryotic cells include hormone-regulated elements (e.g., see Mader, S. and White, J. H. (1993) Proc. Natl. Acad. Sci. USA 90: 5603-5607), synthetic ligand-regulated elements (see, e.g., Spencer, D. M. et al. 1993) Science 262: 1019-1024) and ionizing radiation-regulated elements (e.g., see Manome, Y. Et al. (1993) Biochemistry 32: 10607-10613; Datta, R. et al. (1992) Proc. Natl. Acad. Sci. USA 89: 101410153). Additional tissue-specific or inducible regulatory systems, which may be developed, can also be used in accordance with the invention.

There are a number of techniques known in the art for introducing genetic material into a cell that can be applied to modify a cell of the invention.

In one embodiment, the nucleic acid is in the form of a naked nucleic acid molecule. In this situation, the nucleic acid molecule introduced into a cell to be modified consists only of the nucleic acid encoding the gene product and the necessary regulatory elements.

Alternatively, the nucleic acid encoding the gene product (including the necessary regulatory elements) is contained within a plasmid vector. Examples of plasmid expression vectors include CDM8 (Seed, B. (1987) Nature 329: 840) and pMT2PC (Kaufman, et al. (1987) EMBO J. 6: 187-195).

In another embodiment, the nucleic acid molecule to be introduced into a cell is contained within a viral vector. In this situation, the nucleic acid encoding the gene product is inserted into the viral genome (or partial viral genome). The regulatory elements directing the expression of the gene product can be included with the nucleic acid inserted into the viral genome (i.e., linked to the gene inserted into the viral genome) or can be provided by the viral genome itself.

Naked nucleic acids can be introduced into cells using calcium phosphate mediated transfection, DEAE-dextran mediated transfection, electroporation, liposome-mediated transfection, direct injection, and receptor-mediated uptake.

Naked nucleic acid, e.g., DNA, can be introduced into cells by forming a precipitate containing the nucleic acid and calcium phosphate. For example, a HEPES-buffered saline solution can be mixed with a solution containing calcium chloride and nucleic acid to form a precipitate and the precipitate is then incubated with cells. A glycerol or dimethyl sulfoxide shock step can be added to increase the amount of nucleic acid taken up by certain cells. CaPO₄-mediated transfection can be used to stably (or transiently) transfect cells and is only applicable to in vitro modification of cells. Protocols for CaPO₄-mediated transfection can be found in Current Protocols in Molecular Biology, Ausubel, F. M. et al. (eds.) Greene Publishing Associates, (1989), Section 9.1 and in Molecular Cloning: A Laboratory Manual, 2nd Edition, Sambrook et al. Cold Spring Harbor Laboratory Press, (1989), Sections 16.32-16.40 or other standard laboratory manuals.

Naked nucleic acid can be introduced into cells by forming a mixture of the nucleic acid and DEAE-dextran and incubating the mixture with the cells. A dimethylsulfoxide or chloroquine shock step can be added to increase the amount of nucleic acid uptake. DEAE-dextran transfection is only applicable to in vitro modification of cells and can be used to introduce DNA transiently into cells but is not preferred for creating stably transfected cells. Thus, this method can be used for short-term production of a gene product but is not a method of choice for long-term production of a gene product. Protocols for DEAE-dextran-mediated transfection can be found in Current Protocols in Molecular Biology, Ausubel, F. M. et al. (eds.) Greene Publishing Associates (1989), Section 9.2 and in Molecular Cloning: A Laboratory Manual, 2nd Edition, Sambrook et al. Cold Spring Harbor Laboratory Press, (1989), Sections 16.41-16.46 or other standard laboratory manuals.

Naked nucleic acid can also be introduced into cells by incubating the cells and the nucleic acid together in an appropriate buffer and subjecting the cells to a high-voltage electric pulse. The efficiency with which nucleic acid is introduced into cells by electroporation is influenced by the strength of the applied field, the length of the electric pulse, the temperature, the conformation and concentration of the DNA and the ionic composition of the media. Electroporation can be used to stably (or transiently) transfect a wide variety of cell types and is only applicable to in vitro modification of cells. Protocols for electroporating cells can be found in Current Protocols in Molecular Biology, Ausubel F. M. et al. (eds.) Greene Publishing Associates, (1989), Section 9.3 and in Molecular Cloning: A Laboratory Manual, 2nd Edition, Sambrook et al. Cold Spring Harbor Laboratory Press, (1989), Sections 16.54-16.55 or other standard laboratory manuals.

Another method by which naked nucleic acid can be introduced into cells includes liposome-mediated transfection (lipofection). The nucleic acid is mixed with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J Med. Sci. 298:278; and Gould-Fogerite et al. (1989) Gene 84:429-438.

Naked nucleic acid can also be introduced into cells by directly injecting the nucleic acid into the cells. For an in vitro culture of cells, DNA can be introduced by microinjection. Since each cell is microinjected individually, this approach is very labor intensive when modifying large numbers of cells. However, a situation wherein microinjection is a method of choice is in the production of transgenic animals (discussed in greater detail below). In this situation, the DNA is stably introduced into a fertilized oocyte, which is then allowed to develop into an animal. The resultant animal contains cells carrying the DNA introduced into the oocyte. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BioRad).

Naked nucleic acid can be complexed to a cation, such as polylysine, which is coupled to a ligand for a cell-surface receptor to be taken up by receptor-mediated endocytosis (see for example Wu, G. and Wu, C. H. (1988) J. Biol. Chem. 263: 14621; Wilson et al. (1992) J. Biol. Chem. 267: 963-967; and U.S. Pat. No. 5,166,320). Binding of the nucleic acid-ligand complex to the receptor facilitates uptake of the DNA by receptor-mediated endocytosis. Receptors to which a DNA-ligand complex has targeted include the transferrin receptor and the asialoglycoprotein receptor. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88: 8850; Cristiano et al. (1993) Proc. Natl. Acad. Sci. USA 90: 2122-2126). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Generally, when naked DNA is introduced into cells in culture (e.g., by one of the transfection techniques described above) only a small fraction of cells (about 1 out of 10⁵) typically integrate the transfected DNA into their genomes (i.e., the DNA is maintained in the cell episomally). Thus, in order to identify cells, which have taken up exogenous DNA, it is advantageous to transfect nucleic acid encoding a selectable marker into the cell along with the nucleic acid(s) of interest. Preferred selectable markers include those, which confer resistance to drugs such as G418, hygromycin and methotrexate. Selectable markers may be introduced on the same plasmid as the gene(s) of interest or may be introduced on a separate plasmid.

A preferred approach for introducing nucleic acid encoding a gene product into a cell is by use of a viral vector containing nucleic acid, e.g., a cDNA, encoding the gene product. Infection of cells with a viral vector has the advantage that a large proportion of cells receive the nucleic acid which can obviate the need for selection of cells which have received the nucleic acid. Additionally, molecules encoded within the viral vector, e.g., a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for review see Miller, A. D. (1990) Blood 76: 271). A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions, which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM, which are well known to those skilled in the art. Examples of suitable packaging virus lines include ψCrip, ψCrip, ψ2 and ψAm. Retroviruses have been used to introduce a variety of genes into many different cell types, including epithelial cells endothelial cells, lymphocytes, myoblasts, hepatocytes, bone marrow cells, in vitro and/or in vivo (see for example Eglitis, et al. (1985) Science 230: 1395-1398; Danosand Mulligan (1988) Proc. Natl. Acad. Sci. USA 85: 6460-6464; Wilson et al. (1988) Proc. Natl Acad. Sci USA 85:3014-3018; Armentano et al., (1990) Proc. Natl. Acad. Sci. USA 87: 6141-6145; Huber et al. (1991) Proc. Natl. Acad. Sci. USA 88: 8039-8043; Feri et al. (1991) Proc. Natl. Acad. Sci. USA 88:8377-8381; Chowdhury et al. (1991) Science 254: 1802-1805; van Beusechem et al. (1992) Proc. Natl. Acad. Sci USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 8:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89: 6482-6486), hepatocytes (Herz and Gerard (1993) Proc. Natl. Acad. Sci. USA 90: 2812-2816) and muscle cells (Quantin et al. (1992) Proc. Natl. Acad. Sci. USA 89: 2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Almand and Graham (1986) J. Virol 57: 267). Most replication-defective adenoviral vectors currently in use are deleted for all or parts of the viral E1 and E3 genes but retain as much as 80% of the adenoviral genetic material.

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics In Micro. And Immunol. (1992) 158: 97-129). It is also one of the few viruses that may integrate its DNA into non-dividing cells, and exhibits a high frequency of stable integration (see for example Flotte et al. (1992) Am. J. Respir. Cell. Mol. Biol. 7: 349-356; Samulski et al. (1989) J. Virol. 63:3822-3828; and McLaughlin et al. (1989) J. Virol. 62: 1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA 81: 6466-6470; Tratschin et al. (1985) Mol. Cell Biol. 4: 2072-2081; Wondisford et al. (1988) Mol. Endocrinol. 2:32-39; Tratschin et al. (1984) J. Virol. 51: 611-619; and Flotte et al. (1993) J. Biol. Chem. 268: 3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone.

When the method used to introduce nucleic acid into a population of cells results in modification of a large proportion of the cells and efficient expression of the gene product by the cells (e.g., as is often the case when using a viral expression vector), the modified population of cells may be used without flier isolation or subcloning of individual cells within the population. That is, there may be sufficient production of the gene product by the population of cells such that no further cell isolation is needed. Alternatively, it may be desirable to grow a homogenous population of identically modified cells from a single modified cell to isolate cells, which efficiently express the gene product. Such a population of uniform cells can be prepared by isolating a single modified cell by limiting dilution cloning followed by expanding the single cell in culture into a clonal population of cells by standard techniques.

According to the methods of the present invention, the ex-vivo expanded cells are implanted into an endodermal organ of a subject in need of enhancement thereof. In one embodiment, the subject is a human. As used herein, the phrase “in need thereof” indicates the state of the subject, wherein enhancement of one or more endodermal organs is desirable. Such a state can include, but is not limited to, subjects suffering from primary liver disease such as primary biliary cirrhosis, hepatic cancer, primary sclerosing cholangitis, autoimmune chronic hepatitis, alcoholic liver disease and infectious disease such as hepatitis C, secondary conditions such as the hepatic stage of parasitic infections (helminthes, etc), drug and chemical toxicity, pancreatic diseases such as acute and chronic pancreatitis, hereditary pancreatitis, pancreatic cancer and diabetes. In any of the methods of this aspect of the present invention, the donor and the recipient of the stem and/or progenitor cells can be a single individual or different individuals, for example, allogeneic or xenogeneic individuals. When allogeneic transplantation is practiced, regimes for reducing implant rejection and/or graft vs. host disease, as well know in the art, should be undertaken. Such regimes are currently practiced in human therapy. Most advanced regimes are disclosed in publications by Slavin S. et al., e.g., J Clin Immunol (2002) 22: 64, and J. Hematother Stem Cell Res (2002) 11: 265), Gur H. et al. (Blood (2002) 99: 4174), and Martelli M F et al, (Semin Hematol (2002) 39: 48), which are incorporated herein by reference.

As described hereinabove, the outcome of stern cell differentiation has been shown to be plastic, and sensitive to the presence of, and timing of exposure to a variety of growth factors and combinations thereof. In recent years there have been reports that progenitor cells from adult organs can give rise to unrelated cell types, both in vivo and in culture. It is unclear at present whether these findings represent bona fide “transdifferentiation”, or the persistence of residual immature pluripotent cells in adult tissues (Kohyama et al. 2001; French et al. 2002; Jackson et al. 2002). The most striking example is bone marrow cells from both mice and humans, which have been shown to give rise to a diverse range of cell types from other tissues (Jiang et al. 2002a; Jiang et al. 2002b). While the natural plasticity of tissue stem cells is still being debated, there is evidence that progenitor cells committed to develop into certain tissues can be at least partly reprogrammed with dominant genes that activate a cascade of developmental events. For example, expression of a homeobox transcription factor, Pdx1, which plays key roles in pancreas development and gene expression in mature β cells, in mouse liver cells in vivo (Ferber et al. 2000), and in rat enterocytes in vitro (Kojima et al. 2002), was shown to activate β-cell genes, including insulin. Liver progenitor cells represent an attractive source, since liver and pancreas share embryological origin from the primitive foregut (Tan et al. 1994; Deutsch et al. 2001; Jones et al. 2001). In addition, mature hepatocytes and β cells manifest similarities in gene expression profiles, such as transcription factors, the glucose transporter GLUT2, and the glucose phosphorylating enzyme glucokinase(Kojima et al. 2003; Odom et al. 2004). Progenitor cells cultured from mouse fetal liver were shown to be pluripotent and differentiate in vivo into a number of hepatic, pancreatic, and intestinal cell types (Suzuki et al. 2002, Schwartz et al. J. Clin Invest. 2002;109:1291-1302). Furthermore, adult rat hepatic stem cells, termed oval cells, were shown to differentiate into pancreatic endocrine cells in vitro (Yang et al. 2002). Recently, it was shown that human fetal liver cells transfected with the Pdx1 gene turn on β-cell gene expression (Zalzman et al. 2003). These cells produce and store mature insulin in amounts close to those of normal β cells, and release it in response to physiological glucose concentrations. When transplanted into diabetic immunodeficient mice, these cells restore and maintain euglycemia.

In a recent study (see PCTIL03/00235, to Peled, from which the present application claims priority) the present inventor unexpectedly discovered that ex vivo expanded stem cells differentiate into various cell type, including heart, lung, bone marrow and vascular cells following in vivo administration. Such transdifferentiation of non-endodermally-derived stem and/or progenitor cells into stem cells having an endodermal phenotype is desirable for the ex-vivo preparation of large numbers of expanded cells suitable for transplantation into endodermal organs, according to the methods of the present invention.

Thus, according to one aspect of the present invention there is provided a method of expanding and transdifferentiating a population of non-endodermally derived stem cells into stem cells having an endodermal phenotype, the method effected by (a) obtaining a population of cells comprising stem and/or progenitor cells; (b) culturing the stem and/or progenitor cells ex-vivo under conditions allowing for cell proliferation and, at the same time, culturing said cells under conditions selected from the group consisting of: (i) conditions reducing expression and/or activity of CD38 in the cells; (ii) conditions reducing capacity of the cells in responding to signaling pathways involving CD38 in the cells; (iii) conditions reducing capacity of the cells in responding to retinoic acid, retinoids and/or Vitamin D in the cells; (iv) conditions reducing capacity of the cells in responding to signaling pathways involving the retinoic acid receptor, the retinoid X receptor and/or the Vitamin D receptor in the cells; (v) conditions reducing capacity of the cells in responding to signaling pathways involving PI 3-kinase; (vi) conditions wherein the cells are cultured in the presence of nicotinamide, a nicotinamide analog, a nicotinamide or a nicotinamide analog derivative or a nicotinamide or a nicotinamide analog metabolite; (vii) conditions wherein the cells are cultured in the presence of a copper chelator; (viii) conditions wherein the cells are cultured in the presence of a copper chelate; (ix) conditions wherein the cells are cultured in the presence of a PI 3-kinase inhibitor; thereby expanding the stern and/or progenitor cells while at the same time, substantially inhibiting differentiation of the stem and/or progenitor cells ex-vivo; and (c) inducing enrichment of said stem/progenitor cells for stem cells expressing endodermal cell markers, thereby expanding and transdifferentiating a population of non-endodermal stem cells into stem cells having an endodermal phenotype. Suitable endodermal cell markers, and methods of enrichment for stem and/or progenitor cells expressing endodermal cell markers, are described in detail hereinabove. Additional methods of transdifferentiation of stem cells are disclosed in US Patent Applications 20010033834 to Wilkinson, et al, 20020182728 to Ramiya et al, and 20030185805, 20020068051, and 20020068046 to all Dai et al., which are incorporated by reference as if full set forth herein.

The methods described hereinabove for ex-vivo expanding and transdifferentiating a population of non-endodermally derived stem and/or progenitor cells into stem cells having an endodermal phenotype can result, inter alia, in an expanded population of non-endodermally derived stem cells having an endodermal stem cell phenoptype.

Thus, further according to an aspect of the present invention there is provided a therapeutic ex-vivo cultured population of non-endodermally derived stem and/or progenitor cells expanded and transdifferentiated according to the methods detailed hereinabove, the population comprising a plurality of cells characterized by endodermal cell phenotypic markers such as HNF-3β, GATA1,4 and 6, CK-19, transerythrin, albumin and urea synthesis, cytochrome p450 synthesis, and the presence of binucleated cells, PDX-1, PAX-4, PAX-6, Nkx2.2 and Nkx6.1, insulin I, insulin II, glucose transporter 2, and glucagon, and islet-specific hormones detectable by immunocytochemistry such as insulin, glucagon, and pancreatic polypeptide. Methods of monitoring the expression of these characteristics including, but not limited to immunocytochemistry, dye uptake, FACS analysis, RT/PCR and biological functional analysis, such as indulin, albumin, or urea synthesis are well known in the art.

It will be appreciated, in the context of the present invention, that the therapeutic stem cell population can be provided along with the culture medium containing the hepatic growth factors and/or sodium butyrate, isolated from the culture medium, and combined with a pharmaceutically acceptable carrier. Hence, cell populations of the invention can be administered in a pharmaceutically acceptable carrier or diluent, such as sterile saline and aqueous buffer solutions. The use of such carriers and diluents is well known in the art. Thus, according to another aspect of the present invention, there is provided a pharmaceutical composition comprising cell populations of the invention and a pharmaceutically acceptable carrier.

The ex-vivo population cultured stem cell population of the present invention, expressing endodermal cell phenotypic markers, as described hereinabove, can be used to produce endocrine hormones characteristically secreted by endodermal organs, such as islet cell hormones insulin I, insulin II, glucagon, somatostatin, etc. According to another aspect of the present invention, producing an endocrine hormone is effectd by culturing and transdifferentiating the cells as described hereinabove, and continuing to culture the transdifferentiated cells in the medium, whereby endocrine hormones may be produced. Such media would preferentially include at least one hepatic growth factor and/or sodium butyrate. In one preferred embodiment, the cells are selected from the expanded cell population for cells expressing characteristic endocrine hormones, isolated and further sub-cultured. Induction, isolation and purification of hormones from mammalian cells in culture is well known in the art.

It will be appreciated, that since, as disclosed herein, the ex-vivo expanded stem cells of the present invention can restore normal pancreatic islet function in STZ-diabetic animals, implantation of the ex-vivo expanded cells of the present invention into a subject in need of tissue or cell replacement, can be used for the treatment or prevention of disease of an endodermal organ, such as liver or pancreatic disease. Thus, according to another aspect of the present invention there is provided the abovementioned method used for treating or preventing a liver or pancreatic disease.

According to further features in preferred embodiments of the invention described below the liver disease is selected from the group consisting of primary biliary cirrhosis, hepatic cancer, primary sclerosing cholangitis, autoimmune chronic hepatitis, alcoholic liver disease, infectious hepatitis, parasitic hepatic disease, steatohepatitis and hepatic toxicity.

According to further features in preferred embodiments of the invention described below the pancreatic disease is selected from the group consisting of acute pancreatitis, chronic pancreatitis, hereditary pancreatitis, pancreatic cancer, and diabetes.

Methods of administration of cells for treatment of such diseases by implantation and repopulation of diseased organs are well known in the art (for detailed description of such methods see, for example, U.S. Pat. No. 5,843,156 to Slepian et al, and U.S. Pat. No. 5,670,140 to Sherwin et al, both incorporated by reference as if fully set forth herein.). In a preferred embodiment, the cells are administered by direct implantation into the endodermal organ. In addition to direct implantation of cells, cellular therapy can be also provided via implantation of bioreactors, indwelling and external bio-artificial devices, encapsulated cells, and the like. In one preferred embodiment, the function of the implanted cells is monitored after implantation, by any of the methods described hereinabove. Monitoring of organ function in the implanted subject can be performed periodically, by analysis of markers of organ function in fluids or gas, such as blood, urine, etc, (blood glucose, C-peptide, etc), by analysis in tissue sample from the repopulated organ or by challenge, such as in the Glucose Tolerance Test described hereinbelow. It will be appreciated that frequent monitoring, at intervals of minutes or hours, is desirable soon after implantation, with a decreasing need, at intervals of days, weeks or months, as continued satisfactory function of the repopulated endodermal organ is confirmed.

It has been recently demonstrated (Jang et al., Nat. Cell Biol., adv. online pub. May 9, 2004) that differentiation of hematopoietic stem cells into endodermal cells can be stimulated by co-culturing with injured or damaged liver tissue. Jang et al clearly showed that cell fusion is not required, rather, that specific factors originating within the damaged liver tissue are sufficient to trigger the maturation and differentiation of the stem cells into endodermal cells. Thus, it was not known whether under similar conditions, stem cells capable of restoring pancreatic islet function could also be produced by such co-culture. While reducing the present invention to practice, it was shown, for the first time, that (see Example III) ex-vivo expanded hematopoietic stem cells (CD133+) co-cultured with injured liver tissue efficiently restored pancreatic islet function when transplanted directly into the pancreas of SCID/SCID/bg/bg, STZ-diabetic mice.

Thus, according to further features in preferred embodiments of the invention described below, the step of culturing the stem and/or progenitor cells ex-vivo further includes a step of co-culturing the stem and/or progenitor cells with endodermally-derived organ tissue. The endodermally-derived organ tissue can be healthy, or damaged or injured, as in toxic damage or regeneration following partial organectomy.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document, The procedures therein are believed to be well known in the art and are provided for the convenience of the reader. All the information contained therein is incorporated herein by reference.

### Materials and Experimental Methods

Cells and Processing for Expansion and Transplantation:

Cell source: Cells for expansion and transplantation were from one or more of the following sources:

Hematopoietic stem cells (HSC) or progenitor cells (HPC) from either umbilical cord blood (UCB), G-CSF mobilized peripheral blood (MPB) or bone marrow (BM);

Human stem/progenitor cells derived from either umbilical cord blood (UCB), G-CSF mobilized peripheral blood (MPB) or bone marrow (BM) induced to become enriched with endodermal stem cells (hEndSC rich).

Adult mouse hepatocytes.

Cell cultures of HSC or HPC: Human umbilical cord blood cells were obtained from umbilical cord blood after normal full-term delivery (informed consent was given). MPB, or BM were obtained from donations (informed consent was given). Samples were either used fresh or collected and frozen according to well known cord blood cryopreservation protocol (Rubinstein et al. 1995) within 24 h postpartum for UCB or according to common practice regarding MPB and BM. Prior to cryopreservation, blood was sedimented for 30 minutes on HESPAN Starch hydroxyethyl starch) to remove most RBC. Prior to their use, the cells were thawed in Dextran buffer (Sigma, St. Louis, Mo., USA) containing 2.5% human serum albumin (HSA)(Bayer Corp. Elkhart, Ind., USA) and processed as described hereinbelow. Following thawing, where indicated, the leukocyte-rich fraction was harvested and layered on Ficoll-Hypaque gradient (1.077 g/mL; Sigma Inc, St Louis Mo., USA), and centrifuged at 400×g for 30 minutes. The mononuclear cells in the interface layer was then collected, washed three times, and re-suspended in phosphate-buffered saline (PBS) (Biological Industries) containing 0.5% human serum albumin (HSA) (Bayer Corp. Elkhart, Ind., USA). The CD133⁺ cell fraction was purified as follows: Either the mononuclear cell fraction was subjected to two cycles of immuno-magnetic separation using the “MiniMACS CD133 stem cell isolation kit” (Miltenyi Biotec, Auburn, Calif.) or the unfractionated preparation was isolated on the CliniMACS device using CD133⁺ CliniMACS (Miltenyi Biotec, Auburn, Calif.) reagent, accordingly, following the manufacturer's recommendations. The purity of the CD133⁺ population thus obtained was 92-95%, as evaluated by flow cytometry.

Ex vivo expansion of CD133⁺ in HSC conditions: Purified CD133⁺ cells were cultured in culture bags (American Fluoroseal Co. Gaithersburg, Md., USA) at a concentration of 1×10⁴ cells/ml in alpha minimal essential medium (MEMα) supplemented with 10% FCS containing the following human recombinant cytokines: Thrombopoietin (TPO), interleukin-6 (IL-6), FLT-3 ligand and stem cell factor (SCF), each at a final concentration of 50-150 ng/ml (Perpo Tech, Inc., Rocky Hill, N.J., USA), with 5 μM tetraethylenepentamine (TEPA) (Aldrich, Milwaukee, Wis., USA) and incubated at 37° C. in a humidified atmosphere of 5% CO₂ in air. The cultures were topped up weekly with the same volume of fresh medium, TEPA and growth factors during up to three weeks of expansion.

Ex vivo expansion of CD133⁺ in hEndSC-positive conditions: Purified CD133⁺ cells were cultured in tissue culture flasks (BDFalcon Division, Becton Dickinson and Co, San Jose, Calif., USA) at a concentration of 5×10³ cells/ml in MEMα with 15% FCS, 2 mM L-glutamine, 25 mM HEPES, 100 μL antibiotics (pen/strep), 1 mM 2-mercaptoethanol and 0.5 μM dexamethasone containing the following human recombinant growth/differentiation factors: bFGF, FGF-1 and FGF-2 (each at 20 ng/ml), LIF, HGF, interleukin-6 (IL-6), OSM and stem cell factor (SCF), each at a final concentration of 10-50 ng/ml (Perpo Tech, Inc., Rocky Hill, N.J., USA), as indicated, with 5 μM tetraethylenepentamine (TEPA) (Aldrich, Milwaukee, Wis., USA) and incubated at 37° C. in a humidified atmosphere of 5% CO₂ in air. The cultures were topped up weekly with the same volume of fresh medium, TEPA and growth factors up to three weeks of expansion. Half of the cells were cultured at elevated glucose concentrations 4% (w/v, 400 mg/10 ml) as compared to 1% (Normal Glucose).

Liver damage and coculture. 12-48×10⁴ two-day hend stem cells were co-cultured in a 100 mm tissue culture dish (Corning) separated by a trans-well membrane (pore size 0.4 μm; Corning Costar) from 50 mg of murine liver tissue. At 48 hours, the cells in the upper chamber were recovered and determined to be roughly 80-90% viable. Damaged liver was obtained from mice that had been either exposed to hepatotoxic reagents (acetaminophen at 300 mg per kg, i.p.) or damaged by 40-50% partial hepatectomy 24 hours before the co-culture. The cells were co-cultured in hEndSC medium, as described hereinabove, for 3-48 hours. The cells were then transferred to another flask and/or culture bag without any liver tissue for continuation of culture for additional 17-18 days.

Isolation and Culture of Primary Adult Mouse Hepatocytes:

Intact livers were harvested from 3 week old VLVC female mice (Harlan Laboratories, Jerusalem, Israel), dissected and washed twice with DMEM (Beit Haemek, Israel), incubated with DMEM in the presence 0.05% collagenase for 30 minutes at 37° C., ground and passed through a 200 μm mesh sieve, yielding individual hepatocytes. Cells were washed twice and viability was ascertained with trypan blue. Cells were plated in collagen-coated, 35 mm tissue culture plates at a density of 4-×10⁴ live cells/ml in F12 media (containing 15 mM Hepes, 0.1% glucose, 10 mM sodium bicarbonate, 100 units/ml penicillin-streptomycin, glutamine, 0.5 units/ml insulin, 7.5 m cg/ml hydrocortisone, and 10% fetal bovine serum). Medium was changed after 12 hours, the cells were washed twice with phosphate buffered saline (PBS) and new medium was added. Medium was changed twice a week.

Hepatocytes were also grown in the presence of Epidermal Growth Factor (EGF), Platelet-Derived Growth Factor β chain (PDGF-BB), Fibroblast growth Factors (FGF-4) and Hepatocyte Growth Factor (HGF), at 20-50 ng/ml each, for the entire culturing period according to the method of Schwartz et al. (Schwartz R E, Reyes M, Koodie L, Jiang Y, Blackstad M, Lund T, Lenvik T, Johnson S, Hu W S, Verfaillie C M. Multipotent adult progenitor cells from bone marrow differentiate into functional hepatocyte-like cells. J Clin Invest. 2002; 109 (10): 1291-302). Hepatocytes were also grown in serum free medium according to the method of Runge et al. (Runge D, Runge D M, Jager D, Lubecki K A, Beer Stolz D, Karathanasis S, Kietzmann T, Strom S C, Jungermann K, Fleig W E, Michalopoulos G K. Serum-free, long-term cultures of human hepatocytes: maintenance of cell morphology, transcription factors, and liver-specific functions. Biochem Biophys Res Commun. 2000; 269(1): 46-53).

In all of the above-mentioned hepatocytes culture conditions, cells are grown in the presence or absence of the retinoic acid antagonist AGN 194310 or the transition metal chelator tetraethylenepentamine TEPA) at 10⁻⁵ M.

After a period of 3 weeks, cultures treated with 10⁻⁵ M AGN 194310 or TEPA were detached with 0.25% trypsin, split and replated at a 1:2 ratio. The cells were visualized with Giemsa staining.

Murine hepatocyte cultures supplemented with EGF and HGF were evaluated as primary cultures, or following first and second passages. First passage cultures were grown for 2 weeks, split 1:2 and cells stained 8 days later. Second passage cultures were similarly grown for 2 weeks, split 1:2, and grown for an additional week, then split 1:4 and similarly stained 4 days later.

Histologic Characterization:

Hepatocytes and ex-vivo expanded cells were fixed in methanol directly in their cell culture plates and each procedure performed by standard procedures as outlined below.

Ex-vivo expanded cells and hepatocytes were stained with Giemsa stain, according to manufacturer's instructions (Shandon, Pittsburg, Pa.) for 4 minutes at room temperature, washed in buffer solution for 4 minutes, washed 3-4 times with rinse solution and visualized by light microscopy. Nuclei were stained with hematoxylin (Dako, Carpinteria, Calif.).

Transplanstation of Human Blood-Derived Stem Cells into SCID/SCID/bg/bg Mice

Processing of cells towards transplantation: Following the indicated duration of culture the cells were washed with a PBS solution containing 1% (v/v) HSA human serum albumin) and 2 mM EDTA and were further processed to provide the following different permutations of blood derived stem cells processed for injection: Repurified CD133⁺ cells: CD133⁺ cells were re-purified from the culture media as described hereinabove at different intervals from culturing (i.e., following 3 days, 1 and 3 weeks). In brief, total nucleated cells (TNC) derived from one portion of the culture were subjected to two cycles of immunomagnetic bead separation using the “MiniMACS CD133 Stem Cell isolation kit” (Miltenyi Biotec, Aubun, Calif.). The purity of the CD133⁺ population thus obtained was 95-98%, as evaluated by flow cytometry. TNCs: TNCs derived from the other portion of the culture were subjected to two additional rinses in PBS solution containing 1% (v/v) HSA and 2 mM EDTA. The cells were then transplanted into the mice as described hereinbelow. Repurified hEndSC CD133⁺ cells: CD133⁺ cells were re-purified from the TNCs derived from the hEndSC culture media as described hereinabove at different intervals by immunomagnetic bead separation. hEndSC Normal Glucose: TNCs derived from the normal glucose hEndSC culture were subjected to two additional rinses in PBS solution containing 1% (v/v) HSA and 2 mM EDTA. Control: PBS solution containing 1% (v/v) HSA and 2 mM EDTA

Cell Transplantation into SCID/SCID/bg/bg mice: Mice were bred and maintained at the animal facility of an academic institution in Israel. Six-week-old BLW severe combined immunodeficient (SCID/SCID/bg/bg) female mice (Harlan, Jerusalem) were made hyperglycemic by intraperitoneal injection of streptozotocin (STZ) at 180 mg per gram body weight. When blood glucose levels reached equal to or greater than 300 mg/dl, the mice received the indicated amounts of cells in 0.1 ml of PBS via intra-pancreatic cell tranplantation: Numbers of cells injected were 2×10⁶ cells for CD133⁺ cells and hEndSC CD133⁺ cells; and 20×10⁶ cells for TNCs and hEndSC. Three to seven days after the determination of hyperglycemia the cells were transplanted by intra-pancreatic injection (all mice transplanted on the same day). Briefly, the mice were anesthetized, their skin was exposed just about the location of the pancreas and the peritoneum was gently nicked. The pancreas was then revealed using surgical scissors and tweezers. The cells were injected into the pancreas using a small volume syringe and a 30-gauge needle under visual inspection, using a binocular microscope at X20. Following the injection, a cloud of cells was visible in the pancreas.

Monitoring Glucose and Insulin levels: Blood glucose levels were monitored twice per week in samples obtained from the tail vein of fed mice by using Accutrend strips (Roche Diagnostics, N.J.). Serum insulin and human C-peptide levels were determined by RIA in blood samples obtained from the orbital plexus of fed mice, by using the INSIK-5 and Double Antibody C-Peptide (EUROyDPC, Llanberis, U.K.) kits, respectively, according to the manufacturers' instructions. The human C-peptide kit had 0% cross reactivity with mouse C-peptide.

Histology and Immunohistochemistry

Histology: 6 to 10 weeks after transplantation, the mice were sacrificed, their pancreases were removed and representative sections were fixed in formalin and embedded in paraffin or in frozen tissue embedding gel. Briefly, each pancreas was fixed overnight in 10% buffered formalin, incubated with 30% sucrose in 0.1 M PBS at 4° C., and embedded in paraffin or frozen tissue embedding gel (Fischer Scientific Supply, USA). Serial sagittal cryosections were cut at a thickness of 5 μm and were collected on slides.

Immunohistochemistry: Representative sections were immunostained with anti-human leukocyte antigen (HLA)-DR or ABC antibodies (DAKO, USA), which react specifically with mature human, but not rodent cells. Cells were also co-immunostained for detection of insulin, C-peptide, CD133 and CD45. For insulin immunostaining, sections were first incubated for 10 min. at room temperature in 5% BSA, 5% FBS, and 0.1% Triton X-100, and incubated with guinea pig anti-insulin serum (Linco Research, St. Charles, Mo.) diluted 1:1,000 in blocking solution for 1 hour at room temperature. For CD 133 and CD45 immunostaining, sections were incubated for 10 min at room temperature in 5% BSA, 5% FBS, and 0.1% Triton X-100, and incubated with adequate mouse or rabbit primary antibodies (Miltenyi Biotec for CD133 and DAKO for CD133) diluted 1:1,000 in blocking solution for 1 hour at room temperature. Slides were blocked with 3% normal donkey serum for 10 min at room temperature before incubation with donkey anti-Cy3 (indocarbocyanine) or Cy2 and either anti-guinea pig (insulin), anti-mouse (CD133), or anti-rabbit (CD45) sera DTAF (Jackson ImmunoResearch Laboratories, West Grove, Pa.) for 30 min at room temperature. Slides were then rinsed with PBS and coverslipped with fluorescent mounting medium (Kirkegaard and Perry Laboratories, USA). Fluorescence images were obtained using a Zeiss confocal Epifluorescence microscope equipped with an Optronics TEC-470 CCD camera (Optronics Engineering, Goleta, Calif.).

### Experimental Results

### Example I

### Expansion of Hepatic Stem Cells from Primary Adult Mouse Hepatocyte Culture with TEPA and Retinoic Acid Antagonist AGN 194310

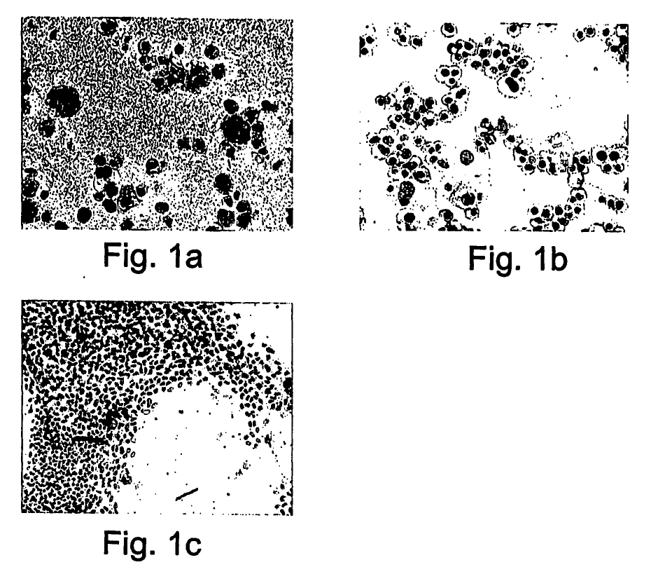

Hepatocytes characteristically grow poorly in culture, with high incidence of cell death. Further, the fraction of hepatic stem cells, known as oval cells, in mature liver is normally very small, increasing usually in response to injury. In order to determine whether hepatic-derived stem cells could be expanded by methods similar to those used for expansion of hematopoietic stem cells, hepatocytes from adult mouse livers, freshly prepared in primary culture, were exposed to the heavy metal chelator tetraethylenepentamine (TEPA, 10 μM) or the retinoic acid agonist AGN 194310 (FIG. 1). Whereas the control cultures treated with culture medium and growth factors EGF and HGF only contained fully matured bi-nucleated hepatocytes (FIG. 1A) that ceased proliferation after a few passages, exposure to TEPA or AGN 194310 clearly induced growth of the cultured hepatocytes, and the appearance of hepatic stem (oval) cells [FIGS. 1B (AGN 194310) and 1C (TEPA)]. Hepatocytes in the treated cultures appeared to proliferate for several passages, even following trypsinization.

Thus, fetal or adult hepatic-derived cells can be expanded using methods proven effective for hematopoietic stem and progenitor cells, such as exposure to TEPA or RAR antagonists. Further, these results indicate that exposure of cultured hepatocytes to TEPA or RAR antagonists results in preferential expansion of the hepatic derived stem (oval) cell fraction of the hepatocyte culture. Thus, such a method is suitable for preparation and expansion of hepatic-derived cells for transplantation into endodermally-derived organs.

### Example II

### Reconstitution of Pancreatic Function in STZ Diabetic Mice by Transplantation of Ex-Vivo Expanded Cord Blood-Derived Stem Cells

In order to determine the efficacy of transplantation of expanded, non-endodermally derived stem or progenitor cells for repopulation of injured endodermal organs and restoration of function therein, human Umbilical Cord Blood cells were expanded ex-vivo with TEPA and transplanted, by direct injection into the pancreas, in STZ-diabetic SCID mice.

As is shown in FIG. 2, euglycemia was completely restored in one mouse (5108P), and partially restored in another mouse (5104P) receiving ex-vivo expanded CD133⁺ cells. Of the four mice receiving cells from the unselected TNC fraction of the ex-vivo expanded cultures two had completely restored euglycemia (5115 and 5116).

Insulin secretion in transplanted STZ diabetic mice was monitored by determining levels of C-peptide, indicative of the maturation of the insulin molecule in the pancreatic islets upon secretion. Table 2 presents the results of C-peptide RIA in peripheral blood samples from the transplanted and control STZ-diabetic mice.

TABLE 2
Presence of human C-peptide (in ng/ml) in serum of mice
transplanted with cord blood derived stem cells.

| Mouse# | ng/ml |
|---|---|
| 5104 | 0.15 |
| 5106 | <0.01 |
| 5108 | 0.34 |
| 5111 | <0.01 |
| 5115 | 0.66 |
| 5116 | 1.26 |
| 5117 | <0.005 |

Note the correlation between the blood glucose values in FIG. 1, and the insulin secretion, as measured by C-peptide, in Table 2 (see, for example, diabetic control 5117 compared with euglycemic 5108 or 5116). Thus, the restoration of euglycemia by transplantation of both purified and unselected ex-vivo expanded UBC into STZ-diabetic mice is the result of restored insulin secretion by the transplanted, non-endodermally derived Cord Blood cells.

The degree of response to a glucose load, after starvation, measured in blood sugar levels over two hours or more, is a sensitive indication of the function of the pancreatic islets. Normal glucose tolerance tests show a peak at 20 or 30 minutes, and a rapid decline to normoglycemia by 1 hour. Slower kinetics of blood glucose uptake following the load is an indication of failure of the pancreas to secrete insulin, as in type I diabetes, or insulin intolerance, as in type II diabetes. FIG. 3 shows the kinetics of glucose uptake in 3 transplanted mice having restored pancreatic function (5108P, 5115 and 5116), as determined by blood glucose (FIG. 2) and C-peptide (Table 2). Note the normal initiation of decline at 30 minutes, indicating insulin release in response to the glucose load, in all three mice. While not wishing to be limited by a single explanation, it is possible that slower kinetics of 5116 could indicate impaired insulin release, and/or a degree of insulin intolerance.

### Example III

### Reconstitution of Pancreatic Function in STZ Diabetic Mice by Transplantation of Ex-Vivo Expanded Cord Blood-Derived hEndSC Co-Cultured for 48 hrs with Injured Liver Tissue

Jang et al (Nat. Cell Biol., adv. online pub. May 9, 2004) have demonstrated that co-culture of hematopoietic stern cells with injured liver cells can stimulate differentiation to endodermal progenitor and, ultimately, liver cells. In order to determine the efficacy of transplantation of expanded, non-endodermally derived stem or progenitor cells for repopulation of injured endodermal organs and restoration of function therein, human Umbilical Cord Blood cells were expanded ex-vivo with TEPA in hEndSC conditions and co-cultured with injured liver tissue for 48 hrs and transplanted, by direct injection into the pancreas, in STZ-diabetic SCID mice.

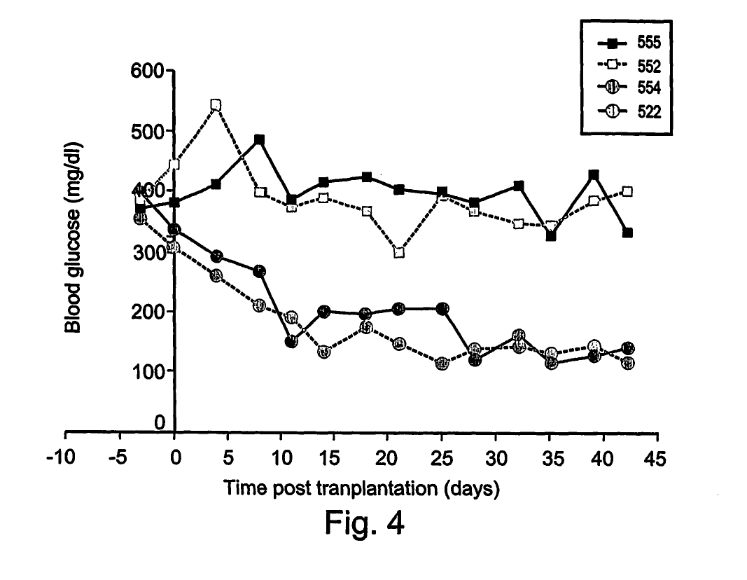

As is shown in FIG. 4, euglycemia was completely restored in the two mice (522, 554) receiving ex-vivo expanded CD133⁺ cells cultured in hEndSC conditions and following 48 hrs co-culture with murine injured liver cells, as compared to the two control mice receiving only PBS (555, and 552).

Thus, hematopoietic stem cells grown under hEnd conditions (endodermal cell growth factors), along with the factors specific to injured liver, have high restorative potential not only of hepatic, but also of pancreatic islet function.

Taken together, the results presented herein show, for the first time, that transplantation of human cord blood cells ex-vivo expanded with TEPA can restore pancreatic islet function in STZ-diabetic mice. Further, full differentiation of the expanded cells prior to implantation is not required for restoration of function, since both selected (CD133⁺) and unselected (TNC) cells were capable of fully restoring pancreatic function in the implanted mice (see, for example, 5108P, 5115 and 5116). Exposure of the cord blood derived stem cells to factors specific to injured liver seemed to enhance the restoration of pancreatic function (see Example III). Thus, the expansion of non-endodermally-derived stern and/or progenitor cells can provide large numbers of cells suitable for repopulation and restoration of function in injured or diseased endodermal organs, such as the liver and pancreas.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

Assady, S., Maor, G., Amit, M., Itskovitz-Eldor, J., Skorecki, K. L. and Tzukerman, M. (2001). “Insulin production by human embryonic stem cells.” Diabetes 50(8): 1691-7.

Ber I, Shternhall K, Pert S, Ohanuna Z, Goldberg I, Barshack I, Benvenisti-Zarum L, Meivar-Levy I, Ferber S. Functional, persistent and extended liver to pancreas transdifferentiation J Biol Chem. 2003

Bonner-Weir, S., Taneja, M., Weir, G. C., Tatarkiewicz, K., Song, K. H., Sharma, A. and O'Neil, J. J. (2000). “In vitro cultivation of human islets from expanded ductal tissue.” Proc Natl Acad Sci USA 97(14): 7999-8004.

Brill S, Zvibel 1, Halpern Z, Oren R. The role of fetal and adult hepatocyte extracellular matrix in the regulation of tissue-specific gene expression in fetal and adult hepatocytes. Eur J Cell Biol. January 2002;81(1):43-50.

Brill S, Zvibel I, Reid L M Expansion conditions for early hepatic progenitor cells from embryonal and neonatal rat livers. Dig Dis Sci. February 1999;44(2):364-71.

Deutsch, G., Jung, J., Zheng, M., Lora, J. and Zaret, K. S. (2001). “A bipotential precursor population for pancreas and liver within the embryonic endoderm.” Development 128(6): 871-81.

Efrat, S. (2001). “Cell therapy approaches for the treatment of diabetes.” Curr Opin Investig Drugs 2(5): 639-42.

Ferber, S., Halkin, A., Cohen, H., Ber, I., Einav, Y., Goldberg, I., Barshack, I., Seijffers, R., Kopolovic, J., Kaiser, N. and Karasik, A. (2000). “Pancreatic and duodenal homeobox gene 1 induces expression of insulin genes in liver and ameliorates streptozotocin-induced hyperglycemia.” Nat Med 6(5): 568-72.

French, S. W., Hoyer, K. K., Shen, R. R. and Teitell, M. A. (2002). “Transdifferentiation and nuclear reprogramming in hematopoietic development and neoplasia.” Immunol Rev 187: 22-39.

Germain L, Blouin M J, Marceau N. Biliary epithelial and hepatocytic cell lineage relationships in embryonic rat liver as determined by the differential expression of cytokeratins, alpha-fetoprotein, albumin, and cell surface-exposed components. Cancer Res. 1988;48(17):4909-18.

Hamazaki T, Iiboshi Y, Oka M, Papst P J, Meacham A M, Zon L I, Terada N. Hepatic maturation in differentiating embryonic stem cells in vitro. FEBS Lett. 2001;497(1):15-9.

Hori, Y., Rulifson, I. C., Tsai, B. C., Heit, J. J., Cahoy, J. D. and Kim, S. K. (2002). “Growth inhibitors promote differentiation of insulin-producing tissue from embryonic stem cells.” Proc Natl Acad Sci USA 99(25): 16105-10.

Jackson, K. A., Majka, S. M., Wulf, G. G. and Goodell, M. A. (2002). “Stem cells: a minireview.” J Cell Biochem Suppl 38: 1-6.

Jiang, Y., Jahagirdar, B. N., Reinhardt, R. L., Schwartz, R. E., Keene, C. D., Ortiz-Gonzalez, X. R., Reyes, M., Lenvik, T., Lund, T., Blackstad, M., Du, J., Aldrich, S., Lisberg, A., Low, W. C., Largaespada, D. A. and Verfaillie, C. M. (2002a). “Pluripotency of mesenchymal stem cells derived from adult marrow.” Nature 418(6893): 41-9.

Jiang, Y., Vaessen, B., Lenvik, T., Blackstad, M., Reyes, M. and Verfaillie, C. M. (2002b). “Multipotent progenitor cells can be isolated from postnatal murine bone marrow, muscle, and brain.” Exp Hematol 30(8): 896-904.

Jones, E. A., Clement-Jones, M., James, O. F. and Wilson, D. I. (2001). “Differences between human and mouse alpha-fetoprotein expression during early development.” J Anat 198(Pt 5): 555-9.

Kakinuma S, Tanaka Y, Chinzei R, Watanabe M, Shimizu-Saito K, Hara Y, Teramoto K, Arii S, Sato C, Takase K, Yasumizu T, Teraoka H.

Human umbilical cord blood as a source of transplantable hepatic progenitor cells. Stem Cells. 2003;21(2):217-27

Kohyama, J., Abe, H., Shimazaki, T., Koinumi, A., Nakashima, K., Gojo, S., Taga, T., Okano, H., Hata, J. and Umezawa, A. (2001). “Brain from bone: efficient “meta-differentiation” of marrow stroma-derived mature osteoblasts to neurons with Noggin or a demethylating agent.” Differentiation 68(4-5): 235-44.

Kojima, H., Fujimiya, M., Matsumura, K., Younan, P., Imaeda, H., Maeda, M. and Chan, L. (2003). “NeuroD-betacellulin gene therapy induces islet neogenesis in the liver and reverses diabetes in mice.” Nat Med 9(5): 596-603.

Kojima, H., Nakamura, T., Fujita, Y., Kishi, A., Fujimiya, M., Yamada, S., Kudo, M., Nishio, Y., Maegawa, H., Haneda, M., Yasuda, H., Kojima, I., Seno, M., Wong, N. C., Kikkawa, R. and Kashiwagi, A. (2002). “Combined expression of pancreatic duodenal homeobox 1 and islet factor 1 induces immature enterocytes to produce insulin.” Diabetes 51(5): 1398-408.

Lumelsky, N., Blondel, O., Laeng, P., Velasco, I., Ravin, R. and McKay, R. (2001). “Differentiation of embryonic stem cells to insulin-secreting structures similar to pancreatic islets.” Science 292(5520): 1389-94.

Mizuguchi T, Hui T, Palm K, Sugiyama N, Mitaka T, Demetriou A A, Rozga J. Enhanced proliferation and differentiation of rat hepatocytes cultured with bone marrow stromal cells. J Cell Physiol. 2001;189(1):106-19.

Miyazaki M, Akiyama I, Sakaguchi M, Nakashima E, Okada M, Kataoka K, Huh N H. Improved conditions to induce hepatocytes from rat bone marrow cells in culture. Biochem Biophys Res Commun. 2002; 298(1):24-30.

Odom, D. T., Zizlsperger, N., Gordon, D. B., Bell, G. W., Rinaldi, N. J., Murray, H. L., Volkert, T. L., Schreiber, J., Rolfe, P. A., Gifford, D. K., Fraenkel, E., Bell, G. I. and Young, R. A. (2004). “Control of pancreas and liver gene expression by HNF transcription factors.” Science 303(5662): 1378-81.

Peled, T., Landau, E., Prus, E., Treves, A. J. and Fibach, E. (2002). “Cellular copper content modulates differentiation and self-renewal in cultures of cord blood-derived CD34+ cells.” Br. J. Haematol. 116(3): 655-661.

Petersen B E, Bowen W C, Patrene K D, Mars W M, Sullivan A K, Murase N, Boggs S S, Greenberger J S, Goff J P. Bone marrow as a potential source of hepatic oval cells. Science. 1999;284(5417):1168-70.

Petersen B E. Hepatic “stem” cells: coming full circle. Blood Cells Mol Dis. 2001; 27(3):590-600.

Rambhatla L, Chiu C P, Kundu P, Peng Y, Carpenter M K. Generation of hepatocyte-like cells from human embryonic stem cells. Cell Transplant. 2003;12(1):1-11.

Ramiya, V. K., Maraist, M., Arfors, K. E., Schatz, D. A., Peck, A. B. and Cornelius, J. G. (2000). “Reversal of insulin-dependent diabetes using islets generated in vitro from pancreatic stem cells.” Nat Med 6(3): 278-82.

Rubinstein, P., Dobrila, L., Rosenfield, R. E., Adamson, J. W., Migliaccio, G., Migliaccio, A. R., Taylor, P. E. and Stevens, C. E. (1995). “Processing and cryopreservation of placental/umbilical cord blood for unrelated bone marrow reconstitution.” Proc. Natl. Acad. Sci. U.S.A 92(22): 10119-10122.

Runge D, Runge D M, Jager D, Lubecki K A, Beer Stolz D, Karathanasis S, Kietzmann T, Strom S C, Jungermann K, Fleig W E, Michalopoulos G K. Serum-free, long-term cultures of human hepatocytes: maintenance of cell morphology, transcription factors, and liver-specific functions. Biochem Biophys Res Commun. 2000; 269(1):46-53.

Schwartz R E, Reyes M, Koodie L, Jiang Y, Blackstad M, Lund T, Lenvik T, Johnson S, Hu W S, Verfaillie C M. Multipotent adult progenitor cells from bone marrow differentiate into functional hepatocyte-like cells. J Clin Invest. 2002; 109(10):1291-302.

Shibata, S., Asano, T., Ogura, A., Hashimoto, N., Hayakawa, J., Uetsuka, K., Nakayama, H. and Doi, K. (1997). “SCID-bg mice as xenograft recipients.” Lab Anim 31(2): 163-8.

Soria, B., Roche, E., Berna, G., Leon-Quinto, T., Reig, J. A. and Martin, F. (2000). “Insulin-secreting cells derived from embryonic stem cells normalize glycemia in streptozotocin-induced diabetic mice.” Diabetes 49(2): 157-62.

Soria B, Skoudy A, Martin F. From stem cells to beta cells: new strategies in cell therapy of diabetes mellitus. Diabetologia. 2001 ;44(4):407-15.

Strom S C, Jirtle R L, Jones R S, Novicki D L, Rosenberg M R, Novotny A, Irons G, McLain J R, Michalopoulos G. Isolation, culture, and transplantation of human hepatocytes. J Natl Cancer Inst. 1982;68(5):771-8.

Suzuki, A., Zheng Yw, Y. W., Kaneko, S., Onodera, M., Fukao, K., Nakauchi, H. and Taniguchi, H. (2002). “Clonal identification and characterization of self-renewing pluripotent stem cells in the developing liver.” J Cell Biol 156(1): 173-84.

Takizawa, Y., Saida, T., Tokuda, Y., Dohi, S., Wang, Y. L., Urano, K., Hioki, K. and Ueyama, Y. (1997). “New immunodeficient (nude-scid, beige-scid) mice as excellent recipients of human skin grafts containing intraepidermal neoplasms.” Arch Dermatol Res 289(4): 213-8.

Tan, C. E. and Moscoso, G. J. (1994). “The developing human biliary system at the porta hepatis level between 29 days and 8 weeks of gestation: a way to understanding biliary atresia. Part 1.” Pathol Int 44(8): 587-99.

Theise N D, Badve S, Saxena R, Henegariu O, Sell S, Crawford J M, Krause D S. Derivation of hepatocytes from bone marrow cells in mice after radiation-induced myeloablation. Hepatology. 2000; 31(1):235-40.

Yamada T, Yoshikawa M, Kanda S, Kato Y, Nakajima Y, Ishizaka S, Tsunoda Y. In vitro differentiation of embryonic stem cells into hepatocyte-like cells identified by cellular uptake of indocyanine green. Stem Cells. 2002;20(2):146-54.

Yang, L., Li, S., Hatch, H., Ahrens, K., Cornelius, J. G., Petersen, B. E. and Peck, A. B. (2002). “In vitro trans-differentiation of adult hepatic stem cells into pancreatic endocrine hormone-producing cells.” Proc Natl Acad Sci USA 99(12): 8078-83.

Zalzman, M., Gupta, S., Giri, R. K., Berkovich, I., Sappal, B. S., Karnieli, O., Zern, M. A., Fleischer, N. and Efrat, S. (2003). “Reversal of hyperglycemia in mice by using human expandable insulin-producing cells differentiated from fetal liver progenitor cells.” Proc Natl Acad Sci USA.

1. A method of enhancing function of an endodermally derived organ in a subject in need thereof, the method comprising:
(a) obtaining a population of cells comprising stem and/or progenitor cells;
(b) culturing said stem and/or progenitor cells ex-vivo under conditions allowing for cell proliferation and, at the same time, culturing said cells under conditions selected from the group consisting of:
(i) conditions reducing expression and/or activity of CD38 in said cells;
(ii) conditions reducing capacity of said cells in responding to signaling pathways involving CD38 in said cells;
(iii) conditions reducing capacity of said cells in responding to retinoic acid, retinoids and/or Vitamin D in said cells;
(iv) conditions reducing capacity of said cells in responding to signaling pathways involving the retinoic acid receptor, the retinoid X receptor and/or the Vitamin D receptor in said cells;
(v) conditions reducing capacity of said cells in responding to signaling pathways involving PI 3-kinase;
(vi) conditions wherein said cells are cultured in the presence of nicotinamide, a nicotinamide analog, a nicotinamide or a nicotinamide analog derivative or a nicotinamide or a nicotinamide analog metabolite;
(vii) conditions wherein said cells are cultured in the presence of a copper chelator;
(viii) conditions wherein said cells are cultured in the presence of a copper chelate;
(ix) conditions wherein said cells are cultured in the presence of a PI 3-kinase inhibitor;
thereby expanding the stem and/or progenitor cells while at the same time, substantially inhibiting differentiation of the stem and/or progenitor cells ex-vivo; and
(c) implanting said cells in an endodermally-derived organ of the subject.

2. The method of claim 1, further comprising monitoring function of said endodermally-derived organ in said subject.

3. The method of claim 1, wherein said stem and/or progenitor cells are derived from a source selected from the group consisting of hematopoietic cells, umbilical cord blood cells, G-CSF mobilized peripheral blood cells, bone marrow cells, hepatic cells, pancreatic cells, neural cells, oligodendrocyte cells, skin cells, gut cells embryonal stem cells, muscle cells, bone cells, mesenchymal cells, chondrocytes and stroma cells.

4. The method of claim 1, wherein step (b) is followed by a step comprising inducing ex-vivo enrichment of said stem/progenitor cells for cells having an endodermal cell phenotype.

5. The method of claim 4, wherein said inducing is effected by providing at least one hepatic growth factor and/or sodium butyrate.

6. The method of claim 5, wherein said hepatic growth factor is selected from the group consisiting of FGF-1, FGF-2, LIF, OSM, HGM, FBS, HGF, EGF, and SCF.

7. The method of claim 1, further comprising the step of selecting a population of stem cells enriched for hematopoietic stem cells.

8. The method of claim 7, wherein said selection is affected via CD34.

9. The method of claim 1, further comprising the step of selecting a population of stem cells enriched for early hematopoietic stem/progenitor cells.

10. The method of claim 9, wherein said selection is affected via CD133.

11. The method of claim 1, wherein step (b) is followed by a step comprising selection of stem and/or progenitor cells.

12. The method of claim 11, wherein said selection is affected via CD 133 or CD 34.

13. The method of claim 1, wherein said endodermally-derived organ is a liver, an intestine or a pancreas.

14. The method of claim 1, wherein said providing said conditions for cell proliferation is effected by providing the cells with nutrients and cytokines.

15. The method of claim 14, wherein said cytokines are selected from the group consisting of early acting cytokines and late acting cytokines.

16. The method of claim 15, wherein said early acting cytokines are selected from the group consisting of stem cell factor, FLT3 ligand, interleukin-6, thrombopoietin and interleukin-3.

17. The method of claim 12, wherein said late acting cytokines are selected from the group consisting of granulocyte colony stimulating factor, granulocyte/macrophage colony stimulating factor and erythropoietin.

18. The method of claim 12, wherein said late acting cytokine is granulocyte colony stimulating factor.

19. The method of claim 1, wherein said subject is a human.

20. The method of claim 1, wherein said stem and/or progenitor cells are genetically modified cells.

21. The method of claim 1, wherein said stem and/or progenitor cells are derived from said subject.

22. The method of claim 1, wherein said inhibitors of PI 3-kinase are wortmannin and/or LY294002.

23. The method of claim 1, wherein step (b) further comprises co-culturing said stem and/or progenitor cells with endodermally-derived organ tissue.

24. A method of expanding and transdifferentiating a population of non-endodermally derived stem cells into stem cells having an endodermal cell phenotype, the method comprising:
(a) obtaining a population of cells comprising stem and/or progenitor cells;
(b) culturing said stem and/or progenitor cells ex-vivo under conditions allowing for cell proliferation and, at the same time, culturing said cells under conditions selected from the group consisting of:
(i) conditions reducing expression and/or activity of CD38 in said cells;
(ii) conditions reducing capacity of said cells in responding to signaling pathways involving CD38 in said cells;
(iii) conditions reducing capacity of said cells in responding to retinoic acid, retinoids and/or Vitamin D in said cells;
(iv) conditions reducing capacity of said cells in responding to signaling pathways involving the retinoic acid receptor, the retinoid X receptor and/or the Vitamin D receptor in said cells;
(v) conditions reducing capacity of said cells in responding to signaling pathways involving PI 3-kinase;
(vi) conditions wherein said cells are cultured in the presence of nicotinamide, a nicotinamide analog, a nicotinamide or a nicotinamide analog derivative or a nicotinamide or a nicotinamide analog metabolite;
(vii) conditions wherein said cells are cultured in the presence of a copper chelator;
(viii) conditions wherein said cells are cultured in the presence of a copper chelate;
(ix) conditions wherein said cells are cultured in the presence of a PI 3-kinase inhibitor;
thereby expanding the stem and/or progenitor cells while at the same time, substantially inhibiting differentiation of the stem and/or progenitor cells ex-vivo; and
(c) inducing enrichment of said stem/progenitor cells for stem cells expressing endodermal cell markers,
thereby expanding and transdifferentiating a population of non-endodermal stem cells into stem cells having an endodermal cell phenotype.

25. The method of claim 24, wherein said stem and/or progenitor cells are derived from a source selected from the group consisting of hematopoietic cells, umbilical cord blood cells, G-CSF mobilized peripheral blood cells, bone marrow cells, neural cells, oligodendrocyte cells, skin cells, gut cells, embryonal stem cells, muscle cells, bone cells, mesenchymal cells, chondrocytes and stroma cells.

26. The method of claim 24, wherein said inducing is effected by providing at least one hepatic growth factor and/or sodium butyrate.

27. The method of claim 26, wherein said hepatic growth factor is selected from the group consisting of FGF-1, FGF-2, LIF, OSM, HGM, FBS, HGF, EGF, and SCF.

28. The method of claim 24, further comprising the step of selecting a population of stem cells enriched for hematopoietic stem cells.

29. The method of claim 28, wherein said selection is affected via CD34.

30. The method of claim 24, further comprising the step of selecting a population of stem cells enriched for early hematopoietic stem/progenitor cells.

31. The method of claim 30, wherein said selection is affected via CD133.

32. The method of claim 24, wherein step (b) is followed by a step comprising selection of stem and/or progenitor cells.

33. The method of claim 32, wherein said selection is affected via CD 133 or CD 34.

34. The method of claim 24, wherein said providing said conditions for cell proliferation is effected by providing the cells with nutrients and cytokines.

35. The method of claim 34, wherein said cytokines are selected from the group consisting of early acting cytokines and late acting cytokines.

36. The method of claim 35, wherein said early acting cytokines are selected from the group consisting of stem cell factor, FLT3 ligand, interleukin-6, thrombopoietin and interleukin-3.

37. The method of claim 35, wherein said late acting cytokines are selected from the group consisting of granulocyte colony stimulating factor, granulocyte/macrophage colony stimulating factor and erythropoietin.

38. The method of claim 37, wherein said late acting cytokine is granulocyte colony stimulating factor.

39. The method of claim 24, wherein said stem and/or progenitor cells are genetically modified cells.

40. The method of claim 24, wherein said endodermal cell markers are selected from the group consisting of insulin, glucagon, somatostatin, pancreatic polypeptide, Pdx-1, pancreatic enzymes, C-peptide, albumin, CK18, CK 19, HNF, THY-1 receptor, c-Met receptor and c-kit.

41. The method of claim 24, wherein step (b) further comprises co-culturing said stem and/or progenitor cells with endodermally-derived organ tissue.

42. A therapeutic ex vivo cultured stem cell population comprising non-endodermally-derived cells expanded and transdifferentiated according to the methods of claim 24.

43. The cell population of claim 42, in a culture medium comprising at least one hepatic growth factor and/or sodium butyrate.

44. The cell population of claim 42, isolated from said medium.

45. A pharmaceutical composition comprising the cell population of claim 43 and a pharmaceutically acceptable carrier.

46. A pharmaceutical composition comprising the cell population of claim 44 and a pharmaceutically acceptable carrier.

47. A method of producing an endocrine hormone comprising the method of claim 24, and further comprising the step of continuing to culture said transdifferentiated cells in said medium, whereby an endocrine hormone may be produced.

48. The method of claim 47, wherein said endocrine hormone is selected from the group consisting of insulin, glucagon and somatostatin.

49. The endocrine hormones produced by the method of claim 47.

50. The method of claim 1, used for treating or preventing a liver or pancreatic disease.

51. The method of claim 50, wherein said liver disease is selected from the group consisting of primary biliary cirrhosis, hepatic cancer, primary sclerosing cholangitis, autoimmune chronic hepatitis, alcoholic liver disease, infectious hepatitis, parasitic hepatic disease, steatohepatitis and hepatic toxicity.

52. The method of claim 50, wherein said pancreatic disease is selected from the group consisting of acute pancreatitis, chronic pancreatitis, hereditary pancreatitis, pancreatic cancer, diabetes.
